SEQUENCE LISTING (9 sequences)

SEQ ID NO: 1 - protein, 9 aa, Artificial Sequence
  Description of Artificial Sequence synthetic peptide
CLLRMXSXC

SEQ ID NO: 2 - protein, 9 aa, Artificial Sequence
  Description of Artificial Sequence synthetic peptide
CLLRMRSIC

SEQ ID NO: 3 - protein, 9 aa, Artificial Sequence
  Description of Artificial Sequence synthetic peptide
CLLRMKSAC

SEQ ID NO: 4 - protein, 9 aa, Artificial Sequence
  Description of Artificial Sequence synthetic peptide
CILRMRSAC

SEQ ID NO: 5 - protein, 9 aa, Artificial Sequence
  Description of Artificial Sequence synthetic peptide
CLIRMRSAC

SEQ ID NO: 6 - protein, 9 aa, Artificial Sequence
  Description of Artificial Sequence synthetic peptide
CLLKMRSAC

SEQ ID NO: 7 - protein, 10 aa, Artificial Sequence
  Description of Artificial Sequence synthetic peptide
CLLRMKASAC

SEQ ID NO: 8 - protein, 9 aa, Artificial Sequence
  Description of Artificial Sequence synthetic peptide
CLLRMRSLC

SEQ ID NO: 9 - protein, 12 aa, Artificial Sequence
  Description of Artificial Sequence synthetic peptide
CVNENGGCEQYC

(19) United States
(12) Patent Application Publication
(10) Pub. No.: US 2005/0048579 A1
(43) Pub. Date: Mar. 3, 2005

(54) Tertiary structures of ICAM-1/LFA-1 modulators

(76) Inventors: Richard S. Larson, Albuquerque, NM (US); Laurel Sillerud, Albuquerque, NM (US)

Correspondence Address: Henry D. Coleman;Coleman Sudol Sapone, P.C., 714 Colorado Avenue, Bridgeport, CT, 06605, US

(21) Appl. No.: 10/886,407

(22) Filed: Jul. 7, 2004

Related U.S. Application Data
(60) Provisional application No. 60/485,343, filed on Jul. 7, 2003.
(60) Provisional application No. 60/495,590, filed on Aug. 15, 2003.

(51) Int. Cl. G01N 33/53; G06F 19/00; G01N 33/48; G01N 33/50

(52) U.S. Cl. 435/7.1; 702/19

(57) ABSTRACT

The invention provides tertiary structures of cyclic nonapeptide ICAM-1 inhibitors and methods of using the tertiary structures to identify additional ICAM-1 inhibitors, particularly non-peptide modulators. The invention also provides methods of treating ICAM-1/LFA-1 mediated diseases comprising administering a non-peptide ICAM-1 modulator of the invention to a patient in need thereof.

### PRIORITY CLAIM

This application claims priority from application Ser. No. 60/485,343, filed Jul. 7, 2003 and application Ser. No. 60/495,590, filed Aug. 15, 2003 under 35 U.S.C. 119(e), in which the contents of both above-mentioned applications are herein incorporated by reference.

### FIELD OF THE INVENTION

The invention relates to computer-generated solution structures of cyclic nonapeptide modulators of ICAM-1 activity. The invention further relates to methods of using the tertiary structures of these ICAM-1 peptide modulators to identify additional ICAM-1 modulators, particularly non-peptide modulators. The invention also relates to methods of treating ICAM-1/LFA-1 mediated diseases comprising administering a non-peptide ICAM-1 modulator of the invention to a patient in need thereof.

### BACKGROUND OF THE INVENTION

Intercellular adhesion molecule-1 (ICAM-1) and leukocyte functional antigen-1 (LFA-1) filed paired receptor/ligand cellular adhesion molecules that are involved in several normal biological functions of leukocytes (Larson and Springer, 1990, Immunological Reviews 114:181-217). Many diseases involving leukocytes are mediated through the interaction of ICAM-1 and LFA-1, including T-cell acute lymphoblastic leukemia (Winter et al., 2001, Br. J. Haematol. 115:862-871), lymphoma metastasis (Umasky et al., 1996, J. Mol. Med. 74:353-363; Zahalka et al., 1993, J. Immunol. 150:4466-4477), ischemia-reperfusion injury (Verrier and Shen, 1993, J. Card. Surg. 8:309-312; Zhao et al., 1997, J. Leukoc. Biol. 62:292-300; Tanaka et al., 1993, J. Immunol. 151:5088-5095; Clark et al., 1991, Stroke 22:877-883), autoimmune diseases (Kawai et al., 1996, Cell. Immunol. 171:262-268; Kimura et al., 1996, Hepatology 24:888-894; Nishikawa et al., 1993, J. Exp. Med. 177: 667-677), solid organ transplant rejection (Cosimi et al., 1990, J. Immunol. 144:4604-4612; Katz et al., 1995, Transplant. Proc. 27:3214; Isobe et al., 1992, Science 255:1125-1127; Kato et al., 1996, Ann. Surg. 223:94-100; Takei et al., 1996, Transplant. Proc. 28:1103-1105), asthma (Wegner et al., 1990, Science 247:456-459), septic shock (Watanabe et al., 1995, Int. Immunol. 7:1037-1046), and diabetic retinopathy (Miyamoto et al., 1999, Proc. Natl. Acad. Sci. U.S.A. 96:10836-10841).

Monoclonal antibodies (mAbs) directed against ICAM-1 have demonstrated therapeutic efficacy in phase I and II clinical trials for several ICAM-1/LFA-1 mediated diseases (Kavanaugh et al., 1996, J. Rheumtatol. 23:1338-1344; Davis et al., 1995, J. Immunol. 254:3525-3537; Haug et al., 1993, Transplantation 55:766-772; Kavanaugh et al., 1994, Arthritis Rheum. 37:992-999). However, the mAbs caused immune reactions (Kavanaugh et al., 1997, Arthritis Rheum. 40:849-853) and secondary physiologic effects (Vuorte et al., 1999, J. Immunol. 102:2353-2357) that precluded clinical use.

In another approach to designing potential therapeutics for treating ICAM-1/LFA-1 mediated diseases, a disulfide-linked cyclic nonapeptide, designated IP01, was produced by phage display directed against ICAM-1 (Shannon et al., 2001, J. Pept. Res. 58:140-150). IP01 proved to be a potent in vitro inhibitor of ICAM-1/LFA-1 dependent cell aggregation. This peptide was modified (designated IP02-K6) and was shown to be a potent in vivo inhibitor of ischemia-reperfusion injury (Merchant et al., 2003, Am. J Physiol. Heart Circ. 284:H1260-1268). Peptide inhibitors have been used therapeutically, but are subject to rapid proteolytic degradation, resulting in short half-lives, and are pH sensitive, which precludes oral administration (Nakanishi et al., 1993, Gene 137:51-56). Intravenous administration of peptides is ineffective because proteases in the blood rapidly clear peptides from circulation. These disadvantages limit the effectiveness of peptides being used as drugs.

However, structural and functional features of peptide inhibitors can be used to generate peptidomimetics, which are more stable and remain in blood circulation longer than peptides, and design or identify non-peptide organic molecules that have the same inhibitory function as the peptide inhibitors. Thus, peptide inhibitors can be used as templates to design clinically effective drugs. There is a need in the art to define solution structures for ICAM-1 peptide inhibitors that can be used to design or identify non-peptide organic molecules and peptidomimetics that can be used clinically to treat ICAM-1/LFA-1 mediated diseases.

### SUMMARY OF THE INVENTION

The invention provides substances that are structurally related to CLLRMXaa₁SXaa₂C (SEQ ID NO:1), where Xaa₁ is R or K and Xaa₂ is I or A. In a specific embodiment, the substance is structurally related to IP01 (a.k.a. Pep04) and/or IP02-K6 (a.k.a. Pep25) and are modulators of ICAM-1 action. These substances can be identified by a screening method of the invention or by rational drug design as described herein. As defined herein a “modulator” is a substance that effects a change in ICAM-1 activity. In a specific embodiment, the “modulator” may inhibit the action of ICAM-1. The substance may have one or more of the following characteristics: forming hydrogen bonds with E34, T35, and K39 of ICAM-1, having hydrophilic interactions with E34 and K77 of ICAM-1, having hydrophobic interactions with M64, L37, and C57 of ICAM-1, comprising a β turn, and/or comprises ICAM-1 binding properties of IP01 and IP02-K6.

In one aspect, the substance may be a peptide variant of SEQ ID NO:1 having the sequence CXaa₁Xaa₂Xaa₃MXaa₄SXaa₅C, wherein Xaa₁ and Xaa₂ are L or I, wherein Xaa₃ and Xaa₄is R or K, Xaa₅ is A, L or I with the proviso that when Xaa₁ and Xaa₂ are L, Xaa₃ is R, Xaa₄ is R or K, Xaa₅ is L. In a specific aspect the peptide variant is selected from the group consisting of: CILRMRSAC (SEQ ID NO:4), CLIRMRSAC (SEQ ID NO:5), CLLKMRSAC (SEQ ID NO:6), CLLRMKASAC (SEQ ID NO:7), CLLRMRSLC (SEQ ID NO:8).

The invention also provides methods of identifying modulators of ICAM-1 by rational drug design comprising the steps of:
(a) employing the tertiary structure of SEQ ID NO:1 to select a potential modulator of ICAM-1, wherein said modulator binds to the ICAM-1 substrate binding site;
(b) obtaining said modulator and
(c) determining whether the potential modulator inhibits ICAM-1.
   Step (a) may be carried out by modeling techniques employing data in Tables 1, 2, 3, 6 and/or 7, or combination of the foregoing ±a root mean square deviation of not more than 2.0 Å from the backbone atoms of the amino acids of SEQ ID NO. 1. In one embodiment, data in Tables 1, 2 and 3 may be employed. In another embodiment, data in Tables 4 and 5 may be employed. The modulator may be obtained using synthetic procedures and may be assayed using procedure described below.

The invention also provides a computer for producing a three dimensional representation of a molecule having the sequence CLLRMXaa₁SXaa₂C (SEQ ID NO:1), where Xaa₁ is R or K and Xaa₂ is I or A comprising (a) a computer-readable data storage medium comprising a data storage material encoded with computer-readable data, wherein said data comprises data in Tables 1, 2, 3, 6 and/or 7, or combination of the foregoing; (b) a memory for storing instructions for processing said computer-readable data; (c ) a central-processing unit coupled to said memory and to said computer-readable data storage medium for processing said computer-machine readable data into said three-dimensional representation; and (d) a display coupled to said central-processing unit for displaying said three-dimensional representation.

In addition, the invention provides methods of identifying modulators of ICAM-1 comprising the steps of: a method of identifying modulators of ICAM-1 comprising: (a) screening for substances having at least one of the characteristics selected from the group consisting of: forming hydrogen bonds with E34, T35, and K39 of ICAM-1, having hydrophilic interactions with E34 and K77 of ICAM-1, have hydrophobic interactions with M64, L37, and C57 of ICAM-1, comprises a β turn, and comprises ICAM-1 binding properties of IP01 and IP02-K6; and (b) determining whether said substance modulates the action of ICAM-1.

In one aspect, the screening comprises using a pharmacophore, virtual screening or related program to find analogous structures of SEQ ID NO:1. In one aspect of the invention, a potential modulator is a non-peptide organic molecule.

The invention also provides a composition comprising an ICAM-1 modulator, wherein the ICAM-1 modulator is structurally related to SEQ ID NO:1 and carrier. The composition may be a pharmaceutical composition. In a specific embodiment, the pharmaceutical composition may be used to treat ICAM-1/LFA-1 mediated diseases.

The invention also provides methods and for treating ICAM-1/LFA-1 mediated diseases comprising administering a therapeutically effective amount of a composition comprising an ICAM-1 modulator, wherein the non-peptide ICAM-1 modulator is structurally related to SEQ ID NO:1.

The invention provides a use for said modulators for the manufacture of medicament for treatment of ICAM/LFA-1 mediated diseases.

Specific preferred embodiments of the present invention will become evident from the following more detailed description of certain preferred embodiments and the claims.

The terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

### BRIEF DESCRIPTION OF THE DRAWINGS

FIG. 1. Fingerprint and α-proton region of the ¹H total correlation spectroscopy (TOCSY) spectrum of inhibitory peptide-01 (IP01). Spin system assignments are labeled and shown with horizontal lines in the fingerprint region and their corresponding α-protons are indicated with bisecting vertical lines. Note that the amino-terminal residue (C1) gives no resonance in the amide region, so its aliphatic assignments are shown with a horizontal line in the up-field region on the F1 axis.

FIG. 2. Sequential assignment of inhibitory peptide-01 (IP01). (A) Amide region of the ¹H nuclear Overhauser effect spectroscopy (NOESY) spectrum of IP01 at mixing time 700 ms showing sequential connectivities. (B) Overlay of ¹H total correlation spectroscopy (TOCSY) fingerprint regions for IP01, IP01 L2-A2, and IP01 R6-A6, distinguishing the L2: L6 and R4: R6 resonances.

FIG. 3. Nuclear Overhauser effect (NOE) build-up schematic. (A) NOE build-up curves of 18:HN inter-residue cross-peaks (18:HN-S7:HN, Hα, Hβ) compared with the scalar cross-peak (I8:γ₁₂-γ₁₁). (B) Fingerprint and amide region of the ¹H NOESY spectrum at mixing time 400 ms showing the inter-residue cross-peaks in the 18:HN segment of the spectrum. (C) Chemical structure of S7-I8 residues with depicted ¹H-resonances highlighted and labeled. (D) Up-field region of the ¹H NOESY spectrum at mixing time 400 ms showing the well-resolved scalar NOE cross-peak between 18:γ₁₂-γ11.

FIG. 4. Ramachandran plots per residue displaying each of the 82 modeled structures. Families A, B, C, D, and six undefined (U) conformations are labeled per legend. Each mark represents the position of a single conformation from each family.

FIG. 5. Difference of selected nuclear magnetic resonance (NMR) data from that of a random coil peptide. Chemical shift differences from the published random coil chemical shifts (Wuthrich, K. (1986) NMR of Proteins and Nucleic Acids. J. Wiley and Sons, New York) are shown in A, while differences from published random coil ³J_{HN-Hα} coupling constants (Asensio, J. L., Martin-Pastor, M. & Jimenez-Barbero, J. (1995) Int. J. Biol. Macromol. 17, 137-148) are shown in B.

FIG. 6. Solvated structures of each conformational family. Ball and stick diagrams represent the lowest backbone root mean squared deviation (RMSD) structure compared with its average structure for each family. The backbone ribbon reflects the per residue RMSD variation (0.6-1.9 Å).

FIG. 7. β-Turns in inhibitory peptide-01 (IP01). Turns with well-defined dihedral angles are labeled by their respective subtype (I, VIII, and U). Unclassifiable turns (U) represent those withdefined dihedral angles for which no turns exist in the current classification scheme. The * represents turns for which multiple dihedral angle ranges exist, thus preventing classification.

FIG. 8. Selected regions of the 500 MHz proton NOESY NMR spectra of IP02-K6. (A) The aliphatic-amide correlation region showing both intraresidue, and interresidue cross peaks. Several of the interresidue peaks are labeled with the contributing spin pairs. (B) The amide region of the NOESY spectrum at a mixing time of 400 ms showing all sequential amide connectivities; labels are shown for amide proton cross-peaks from L2-L3, L3-R4, M5-K6, and S7-A8.

FIG. 9. Differences of NMR data for IP02-K6 from that of a random coil peptide. (A) Chemical shift differences from the published random coil chemical shifts (Laskowski, R. A., MacArthur, M. W., Moss, D. S., & Thornton. J. M. (1993). PROCHECK: a program to check the stereochemical quality of protein structures. Journal of Applied Crystallography 26, 283-291). Significant shift differences are noted for the amide protons of L2, M5, A8 and C9. (B) Differences from published random coil coupling constants (McLachlan, A. D. (1982). Rapid Comparison of Protein Structures. Acta Crystallographica Section A-Foundations of Crystallography 38, 871-873). Significant coupling constant differences are seen for M5, K6, A8 and C9. The chemical shift and coupling constant differences are largest in the functionally-important portion of the molecule from M5-C9.

FIG. 10. Solution conformation of IP02-K6. (A) Stick figure representations of the 16 low-energy, low-RMSD structures. The disulfide-linked cysteines are not shown for clarity. (B) Ball and stick diagram of the lowest energy structure. The backbone ribbon reflects the per residue backbone RMSD variation (0.3 to 0.6 Å). (C) Solvent accessible surface and hydrophobicity analysis for IP02-K6. The surface accessible to water (molecular radius=1.4 Å) is displayed. The molecule is rotated 90 degrees clockwise from A and B.

FIG. 11. Comparison of the solution (white) and ICAM-1-docked structure of IP02-K6. The only major difference between these two forms is seen to lie in the conformations of the side chains.

FIG. 12. Docking of IP02-K6 onto the surface of ICAM-1. IP02-K6 adopts a kinked structure so that the backbone in the region of Met-5 to Ser-7 can drape over P36. Note the position of the methyl group from Alanine-8 in the depression to the right of E59. The residues of IP02-K6 are labeled with three-letter amino acid abbreviations, while those from ICAM-1 are labeled with single-letter amino acid codes.

FIG. 13. Docking of IP02-K6 unto the surface of ICAM-1 compared to the sites occupied during the interaction of LFA-1 with ICAM-1. The orientation of ICAM-1 is 90 degrees counterclockwise from that in FIG. 12.

FIG. 14. The LFA-1 binding site on ICAM-1 deduced by mapping the mutation data (Shimaoka, M., Xiao, T., Liu, J. H., Yang, Y., Dong, Y., Jun, C. D., McCormack, A., Zhang, R., Joachimiak, A., Takagi, J., Wang, J. H., & Springer, T. A. (2003). Structures of the alpha L I domain and its complex with ICAM-1 reveal a shape-shifting pathway for integrin regulation. Cell 112, 99-111) onto the surface of ICAM-1. The ICAM-1 binding site is derived from residues 28-41 of the C and D strands as well as residues 63-81 of the F and G strand (Pontius, J., Richelle, J., & Wodak, S. J. (1996) Deviations from Standard Atomic Volumes as a Quality Measure for Protein Crystal Structures. J. Mol. Biol. 264,121-136).

### DETAILED DESCRIPTION OF THE PREFERRED EMBODIMENTS

IP01 (CLLRMRSIC; SEQ ID NO:2) is a disulfide linked cyclic peptide that binds to ICAM-1 and blocks binding to its counter-structure, leukocyte functional antigen-1 (LFA-1) (Shannon et al., 2001, J. Pept. Res. 58:140-150). IP02-K6 (CLLRMKSAC; SEQ ID NO:3) is a derivative of IP01 produced by single homologous amino acid substitutions. IP02-K6 is a more potent inhibitor of ICAM-1 than IP01 (Merchant et al., 2003, Am. J Physiol. Heart Circ. 284:H1260-1268).

As described in the Examples below, the inventors used two-dimensional nuclear magnetic resonance (NMR) restraints and molecular modeling to produce a solution model of IP01 and IP02-K6. The inventors also used distance and dihedral angle restraints, generated from Overhauser effect spectroscopy (NOESY) and one-dimensional NMR to generate an ensemble of structures using distance geometry (DG) and simulated annealing (SA). The restrained molecular dynamics simulations provided evidence that IP01 exists in three interconverting conformation families, designated A, B, and C. The modeling data further showed that the interconversion occurred as follows: A⇄C⇄B, but not A⇄B, indicating that conformation C is the common intermediate in the interconversion process. In addition, as shown in the Examples, IP02-K6 shares many similarities with conformation C, which was also identified as the dominant conformation for IP01.

The Examples below show that the tertiary structure of IP02-K6 comprises a β-turn between residues R4 and S7. The β-turn was found to have a maximum dihedral angle variation of 60°, which has not been previously reported in the classification of β-turns. In addition, hydrophilic interactions occurred between ICAM-1 residues E34 and IP02-K6 residue K6 and between ICAM-1 residue K77 and IP02-K6 residue C1. Hydrophobic interactions were found between ICAM-1 residue M64 and IP02-K6 residue M5, and between ICAM-1 residues L37 and V57 and IP02-K6 residue A8. Residues L2 and L3 of IP02-K6 were flexible and interacted with ICAM-1 residues E34, K77, T35, P36, and M64. All seven of the ICAM-1 residues that are known to be critical for LFA-1 binding were involved in binding to IP02-K6.

In one embodiment, the invention provides substances that are structurally related to SEQ ID NO:1, particularly IP01 (SEQ ID NO:2) and IP02-K6 (SEQ ID NO:3) and are modulators of ICAM-1. In one embodiment, the non-peptide molecule is a non-peptide organic molecule. In other embodiments, the non-peptide is a peptide analog (e.g. a peptidomimetic).

In one embodiment, an ICAM-1 modulator of the invention can be identified by a screening method of the invention or by rational drug design as described herein.

As used herein, a modulator is “structurally related” to SEQ ID NO:1 if the modulator comprises structural characteristics that mimic (particularly in terms of ICAM-1 binding properties or in tertiary space structure) the peptide structures of SEQ ID NO:1, particularly, IP01 (SEQ ID NO:2) and IP02-K6 (SEQ ID NO:3), or portions (e.g. fragments) thereof, as described herein. In one embodiment, a structurally related non-peptide of the invention comprises a β turn, preferably a β turn that has a maximum dihedral angle of 60°. In another embodiment, the structurally related non-peptide of the invention can form hydrogen bonds with E34, T35, and K39 of ICAM-1, has hydrophilic interactions with E34 and K77 of ICAM-1, and/or has hydrophobic interactions with M64, L37, and C57 of ICAM-1.

The term “non-peptide organic molecule” as used herein encompasses a naturally-occurring or synthetically generated organic compound and includes peptide analogs. Peptide analogs are commonly used in the pharmaceutical industry as non-peptide drugs with properties analogous to those of the template peptide. These types of non-peptide compound are termed “peptide mimetics” or “peptidomimetics.” A peptidomimetic that has been biologically engineered to mimic the behavior of naturally occurring peptides is more stable than the template peptide and remains in the blood stream longer. Such qualities make them more useful as therapeutic agents than template peptides. See, for example, Fauchere, 1986, Adv. Drug Res. 15:29; Veber and Freidinger, 1985, TINS p.392; and Evans et al., 1987, J. Med. Chem. 30:1229, which are incorporated herein by reference for any purpose.

Such compounds are often developed with the aid of computerized molecular modeling. Peptide mimetics that are structurally similar to therapeutically useful peptides, such as IP01 and IP02-K6, can be used to produce a similar therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used in certain embodiments to generate more stable peptides. In addition, constrained peptides comprising a consensus sequence or a substantially identical consensus sequence variation may be generated by methods known in the art (Rizo and Gierasch, 1992, Ann. Rev. Biochem., 61:387, incorporated herein by reference for any purpose).

In one embodiment, the invention provides computer-generated models of the solution structures of SEQ ID NO:1, particularly, IP01 and IP02-K6. As discussed below, the NMR determined solution conformations of IP02-K6 and conformation C of IP01 reflected the ICAM-1 bound conformation of IP02-K6 and the most active ICAM-1 bound conformation of IP01. Thus, in a particular embodiment, the computer-generated models of the solution structures provided herein for IP00 and IP02-K6 can be used in rational drug design to generate further candidate compounds that will bind to ICAM-1 and serve as ICAM-1 modulators. In particular, the invention provides a computer for producing a three dimensional representation of a molecule having the sequence CLLRMXaa₁SXaa₂C (SEQ ID NO:1), where Xaa₁ is R or K and Xaa₂ is I or A, comprising (a) a computer-readable data storage medium comprising a data storage material encoded with computer-readable data, wherein said data comprises data in Tables 1, 2, 3, 6 or 7, or combination of the foregoing; (b) a wording memory for storing instructions for processing said computer-readable data; (c ) a central-processing unit coupled to said working memory and to said computer-readable data storage medium for processing said computer-machine readable data into said thre-dimensional representation; and (d) a display coupled to said central-processing unit for displaying said three-dimensional representation.

In one embodiment, a method of the invention for identifying modulators of ICAM-1 by rational drug design comprises the steps of: (a) designing a potential modulator for ICAM-1 that will bind to the ICAM-1 substrate binding site based upon the tertiary structure of IP01 (CLLRMRSIC; SEQ ID NO:2) or IP02-K6 (CLLRMKSAC; SEQ ID NO:3); (b) synthesizing the modulator; and (c) determining whether the potential modulator modulates the action of ICAM-1. In one aspect of the invention, a potential modulator is a non-peptide organic molecule.

As used herein, the term “rational drug design” refers to a process wherein structural information about a IP01:ICAM-1 interaction and IP02-K6:ICAM-1 interaction is used to design and propose modified ICAM-1 modulator candidate compounds (i.e. potential modulators) possessing the same or improved fit with ICAM-1/LFA-1 binding site in terms of geometry and chemical complementarity and also the same or improved biological and pharmaceutical properties compared with IP01 and IP02-K6. In a preferred embodiment, such candidate compounds are peptide analogs (e.g. peptidomimetics) or synthetic organic molecules.

In another embodiment, using techniques known in the art and those described herein, solution structures of other ICAM-1 modulators that are disulfide-linked cyclic nonapeptides bound to ICAM-1 can be generated and used in a program of rational drug design as described herein. Such cyclic peptide modulators of ICAM-1 are described, for example, in U.S. patent application Ser. No. 09/483,550, filed Jan. 14, 2000, and U.S. patent application Ser. No. 09/760,599, filed Jan. 16, 2001 (which are both hereby incorporated by reference).

Various methods of structure-based drug design are disclosed in the art, for example, in Maulik et al., 1997, Molecular Biotechnology: Therapeutic Applications and Strategies, Wiley-Liss, Inc., which is incorporated herein by reference in its entirety. Maulik et al. disclose, for example, methods of directed design, in which a user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

Several computer programs are known in the art that can be used in designing a potential modulator of the invention, including, but not limited to:
GRID (Goodford, 1985, J. Med. Chem. 28:849-857, which is a program that determines probable interaction sites between probes with various functional group characteristics and the macromolecular surface, can be used to analyze the surface sites to determine structures of similar inhibiting proteins or molecules. The GRID calculations, with suitable inhibiting groups on molecules (e.g., protonated primary amines) as the probe, are used to identify potential hotspots around accessible positions at suitable energy contour levels. GRID is available from Oxford University, Oxford, UK;
MCSS (Miranker and Karplus, 1991, Proteins: Structure, Function and Genetics 11:29-34). MCSS is available from Molecular Simulations, Burlington, Mass.
AUTODOCK (Goodsell and Olsen, 1990, Proteins: Structure, Function, and Genetics 8:195-202). AUTODOCK is available from Scripps Research Institute, La Jolla, Calif.;
DOCK (Kuntz et al., 1982, J. Mol. Biol. 161: 269-288). The program DOCK may be used to analyze an active site or ligand binding site and suggest ligands with complementary steric properties. DOCK is available from University of California, San Francisco, Calif.;
ALADDIN (Van Drie et al., 1989, J. Comp-Aided Mol. Des. 3:225);
CLIX (Davie and Lawrence, 1992, Proteins 12:31-41);
GROUPBUILD (Rotstein and Murcko, 1993, J. Med. Chem. 36:1700);
GROW (Moon and Howe, 1991, Proteins 11:314);
LUDI (Bohm, 1992, J. Comp. Aid. Molec. Design 6:61-78; and Rotstein and Murcko, 1992, J. Med. Chem. 36:1700-1710). LUDI is available from Biosym Technologies, San Diego, Calif.;
LEGEND (Nishibata and Itai, 1991, Tetrahedron 47:8985). LEGEND is available from Molecular Simulations, Burlington, Mass.; and
LeapFrog (available from Tripos Associates, St. Louis, Mo.).

Other molecular modeling techniques can also be used in accordance with the invention, including, but not limited to, Cohen et al., 1990, J. Med Chem. 33:883-894; Navia and Murcko, 1992, Current Opinions in Structural Biology 2:202-210; and Jorgensen, 1998, “BOSS-Biochemical and Organic Simulation System” in the Encyclopedia of Computational Chemistry (P. V. R. Schleyer, ed.) Wiley & Sonstra., Athens, U.S.A. 5:3281-3285).

In one embodiment, it may be possible during modeling to introduce into a potential modulator chemical moieties that may be beneficial for a molecule that will be administered as a pharmaceutical. For example, it may be possible to introduce into or omit from the potential modulator, chemical moieties that may not directly affect binding of the modulator to the ICAM-1 but that contribute, for example, to the overall solubility of the modulator in a pharmaceutically acceptable carrier, the bioavailability of the modulator and/or the toxicity of the modulator. Considerations and methods for optimizing the pharmacology of the modulators of interest can be found, for example, in “Goodman and Gilman's The Pharmacological Basis of Therapeutics,” 1985, Eighth Edition (Goodman Gilman, Rall, Nies, & Taylor, eds.), Pergaman Press; Jorgensen and Duffy, 2000, Bioorg. Med. Chem. Lett. 10:1155-1158.

Also, the computer program “Qik Prop” can be used to provide rapid predictions for physically significant descriptions and pharmaceutically-relevant properties of an organic molecule of interest.

Potential modulators can also be selected based on their structural similarity to IP01 and IP02-K6 by systematically modifying a structural analog with computer modeling programs. For example, such analysis has been described for developing HIV protease modulators (Lam et al., 1994, Science 263:380-384; Wlodawer et al., 1993, Ann. Rev. Biochem. 62:543-585; Appelt, 1993, Perspectives in Drug Discovery and Design 1:23-48; Erickson, 1993, Perspectives in Drug Discovery and Design 1:109-128).

A potential modulator designed using computer modeling can be obtained from a commercial library of chemicals or synthesized de novo. Appropriate methods of chemical synthesis include medicinal chemistry and combinatorial chemistry techniques known to those of skill in the art (see, for example, Advanced Organic Chemistry 2^{nd} edition (J. March) 1977, McGraw-Hill New York and B. A. Bunin, The Combinatorial Index, 1998, Academic Press).

A potential modulator can be screened for binding activity in one of many standard binding assays, such as, for example, a radioligand receptor binding assay on a solid support, or a fluorescence-polarization assay conducted in solution (See for example, Immune and Receptor Assays in Theory and in Practice, Patrick Englebienne, CRC Press 2000).

Determining whether a potential modulator alters (e.g., inhibits) the action of ICAM-1 can be accomplished using cell-based assays or in animal model systems that mimic ICAM-1/LFA-1 mediated disease states. For example, a potential modulator can be tested for its ability to inhibit ICAM-1/LFA-1 mediated cell aggregation using cell aggregation assays as described, for example, in Shannon et al., 2001, J. Pept. Res. 58:140-150; Larson et al., 1997, Blood 90:2747-2756; and Rothlein and Springer, 1986, J. Exp. Med. 163:1132-1149. Alternatively, a potential modulator can be administered to a mouse that is genetically engineered to have an ICAM-1/LFA-1 mediated disease.

In another embodiment, a method of the invention for identifying ICAM-1 modulators comprises the steps of: (a) screening for organic compounds that are structurally related to IP01 (SEQ ID NO:2) or IP02-K6 (SEQ ID NO:3); and (b) determining whether the potential modulator modulates the activity of ICAM-1. In one aspect, the screening comprises using a pharmacophore or related program. Determining the activity of a potential modulator can be accomplished as described above.

The concept of the pharmacophore has been well described in the literature (see, for example, Mayer et al., 1987, J. Comp. Aided Molec. Design 1:3-16; Hopfinger and Burke, 1990, Concepts and Applications of Molecular Similarity, M. A. Johnson and G. M. Maggiora, ed., Wiley). In one embodiment, a pharmacophore of the invention is generated based on the most important common structural features of IP01 and IP02-K6. For example, the pharmacophore would have a β turn that has a maximum dihedral angle variation of 60°. As used herein, “pharmacophore computer programs” encompass software used for computational mining of three-dimensional (3-D) molecular databases to identify compounds that are structurally similar to IP01 and IP02-K6 and can inhibit ICAM-1 activity.

Pharmacophore computer programs that can be used in a method of the invention include, but are limited to:
DISCO (Abbot Laboratories, Abbot Park, Ill.);
Catalyst (Bio-CAD Corp., Mountain View, Calif.); and
Chem DBS-3D (Chemical Design Ltd., Oxford, U.K.).

Databases of chemical structures are available from, for example, Cambridge Crystallographic Data Center (Cambridge, U.K.) and Chemical Abstracts Service (Columbus, Ohio).

Once a potential modulator is identified using a method of the invention, the potential modulator can be examined through the use of computer modeling using a docking program such as GRAM, DOCK or AUTODOCK (Dunbrack et al., 1997, Folding & Design 2:R27-42). This procedure can include computer fitting of candidate compounds to the LFA-1 binding site to ascertain how well the shape and the chemical structure of the potential modulator will complement the binding site. (Bugg et al., 1993, Scientific American December:92-98; West et al., 1995, TIPS 16:67-74). Computer programs can also be used to estimate the attraction, repulsion and steric hindrance of the two binding partners (i.e. the ligand-binding site and the candidate compound).

In another embodiment of the invention, structural analogs of IP01 and IP02-K6 that successfully modulate ICAM-1 can be systematically modified by computer modeling programs to enhance binding properties. For example, such compounds can be modified to satisfy criteria associated with chemical complementarity, such as hydrogen bonding, ionic interactions or Van der Waals interactions.

Once a candidate compound is identified it can be either selected from a library of chemicals as are commercially available or, alternatively, the candidate compound can be synthesized de novo. De novo synthesis of one or even a relatively small group of specific compounds is known in the art of drug design.

A candidate compound can be placed into a standard binding assay with ICAM-1 or a fragment of ICAM-1 that comprises the LFA-1 binding site to determine if the compound can bind ICAM-1. A candidate compound can also be used in an ICAM-1 activity assay as described herein to determine if the compound can inhibit ICAM-1 activity.

The invention also provides a composition, particularly a pharmaceutical composition comprising an ICAM-1 modulator, wherein the ICAM-1 modulator is structurally related to SEQ ID NO:1, particularly IP01 and IP02-K6. In a particular embodiment, the modulator is a non-peptide ICAM-1 modulator which can be an organic molecule or a peptide analog, as defined above. In one embodiment, the non-peptide modulator can form hydrogen bonds with E34, T35, and K39 of ICAM-1, can have hydrophilic interactions with E34 and K77 of ICAM-1, comprises a β turn, preferably a β turn that has a maximum dihedral angle variation of 60° and/or can have hydrophobic interactions with M64, L37, and C57 of ICAM-1.

The invention also provides methods and compositions for treating ICAM-1/LFA-1 mediated diseases comprising administering a therapeutically effective amount of a composition of the invention.

The term “ICAM-1/LFA-1 mediated disease” encompasses any medical condition or disorder associated with increased ICAM-1 expression and/or activity including, but not limited to, T-cell acute lymphoblastic leukemia, lymphoma metastasis, ischemia-reperfusion injury, autoimmune diseases, solid organ transplant rejection, asthma, septic shock, and diabetic retinopathy.

In preferred embodiments, compositions of the invention can comprise a therapeutically effective amount of one or a plurality of ICAM-1 modulators of the invention together with a diluent, carrier, solubilizer, emulsifier, preservative and/or adjuvant. In a particular embodiment, the diluent, carrier, solubilizer, emulsifier, preservative and/or adjuvant is a pharmaceutically acceptable formualtion material. Preferably, acceptable formulation materials are nontoxic to recipients at the dosages and concentrations employed.

In certain embodiments, acceptable formulation materials preferably are nontoxic to recipients at the dosages and concentrations employed.

In certain embodiments, the composition and in a specific embodiment, pharmaceutical composition, may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See REMINGTON'S PHARMACEUTICAL SCIENCES, 18^{th} Edition, (A. R. Gennaro, ed.), 1990, Mack Publishing Company.

In certain embodiments, the optimal composition, particularly pharmaceutical composition, will be determined by one skilled in the art depending upon, for example, the intended route of administration, delivery format and desired dosage. See, for example, REMINGTON'S PHARMACEUTCAL SCIENCES, supra. In certain embodiments, such compositions may influence the physical state, stability, rate of ill vivo release and rate of in vivo clearance of the ICAM-1 modulator compositions of the invention.

In certain embodiments, the primary vehicle or carrier in a composition, particularly, pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In preferred embodiments, pharmaceutical compositions comprise Tris buffer of about pH 7.0-8.5, or acetate buffer of about pH 4.0-5.5, and may further include sorbitol or a suitable substitute therefor. In certain embodiments of the invention, ICAM-1 modulator compositions may be prepared for storage by mixing the selected composition having the desired degree of purity with optional formulation agents (REMINGTON'S PHARMACEUTICAL SCIENCES, supra) in the form of a lyophilized cake or an aqueous solution. Further, in certain embodiments, the ICAM-1 modulator product may be formulated as a lyophilizate using appropriate excipients such as sucrose.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art.

The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the therapeutic compositions for use in this invention may be provided in the form of a pyrogen-free, parenterally acceptable aqueous solution comprising the desired non-peptide ICAM-1 modulator of the invention in a pharmaceutically acceptable vehicle. A particularly suitable vehicle for parenteral injection is sterile distilled water in which the non-peptide ICAM-1 modulator of the invention is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the desired ICAM-1 modulator.

Compositions of the invention can be formulated for inhalation. In these embodiments, ICAM-1 modulators of the invention are advantageously formulated as a dry, inhalable powder. In preferred embodiments, non-peptide ICAM-1 modulator of the invention inhalation solutions may also be formulated with a propellant for aerosol delivery. In certain embodiments, solutions may be nebulized. Pulmonary administration and formulation methods therefore are further described in International Patent Application No. PCT/US94/001875, which is incorporated by reference and describes pulmonary delivery of chemically modified proteins.

It is also contemplated that formulations can be administered orally. ICAM-1 modulators that are administered in this fashion can be formulated with or without carriers customarily used in the compounding of solid dosage forms such as tablets and capsules. In certain embodiments, a capsule may be designed to release the active portion of the formulation at the point in the gastrointestinal tract when bioavailability is maximized and pre-systemic degradation is minimized. Additional agents can be included to facilitate absorption of the non-peptide ICAM-1 modulator of the invention. Diluents, flavorings, low melting point waxes, vegetable oils, lubricants, suspending agents, tablet disintegrating agents, and binders may also be employed.

A composition, particularly pharmaceutical composition of the invention is preferably provided to comprise an effective quantity of one or a plurality of ICAM-1 modulators of the invention in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional compositions will be evident to those skilled in the art, including formulations involving ICAM-1 modulators of the invention in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g. films, or microcapsules. Sustained release matrices may include polyesters, hydrogels, polylactides (as disclosed in U.S. Pat. No. 3,773,919 and European Patent Application Publication No. EP 058481, each of which is incorporated by reference), copolymers of L-glutamic acid and gamma ethyl-L-glutamate (Sidman et al., 1983, Biopolymers 22:547-556), poly (2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., supra) or poly-D(−)-3-hydroxybutyric acid (European Patent Application Publication No. EP 133,988). Sustained release compositions may also include liposomes that can be prepared by any of several methods known in the art. See e.g., Eppstein et al., 1985, Proc. Natl. Acad. Sci. USA 82:3688-3692; European Patent Application Publication Nos. EP 036,676; EP 088,046 and EP 143,949, incorporated by reference.

Compositions, particularly, pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

The invention also provides kits for producing a single-dose administration unit. The kits of the invention may each contain both a first container having a dried protein and a second container having an aqueous formulation. In certain embodiments of this invention, kits containing single and multi-chambered pre-filled syringes (e.g., liquid syringes and lyosyringes) are provided.

The effective amount of a pharmaceutical composition, which comprises an ICAM-1 modulator of the invention and is to be employed therapeutically, will depend, for example, upon the therapeutic context and objectives. One skilled in the art will appreciate that the appropriate dosage levels for treatment will vary depending, in part, upon the molecule delivered, the indication for which the non-peptide ICAM-1 modulator of the invention is being used, the route of administration, and the size (body weight, body surface or organ size) and/or condition (the age and general health) of the patient. In certain embodiments, the clinician may titer the dosage and modify the route of administration to obtain the optimal therapeutic effect. A typical dosage may range from about 0.1 μg/kg to up to about 30 mg/kg or more, depending on the factors mentioned above. In preferred embodiments, the dosage may range from 0.1 μg/kg up to about 30 mg/kg; more preferably from 1 μg/kg up to about 30 mg/kg; or even more preferably from 5 μg/kg up to about 30 mg/kg.

Dosing frequency will depend upon the pharmacokinetic parameters of the particular non-peptide ICAM-1 modulator in the formulation used. Typically, a clinician administers the composition until a dosage is reached that achieves the desired effect. The composition may therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data.

The route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions may be administered by bolus injection or continuously by infusion, or by implantation device.

The composition also may be administered locally via implantation of a membrane, sponge or another appropriate material onto which the desired molecule has been absorbed or encapsulated. In certain embodiments, where an implantation device is used, the device may be implanted into any suitable tissue or organ, and delivery of the desired molecule may be via diffusion, timed-release bolus, or continuous administration.

### EXAMPLES

The following examples, including the experiments conducted and results achieved are provided for illustrative purposes only and are not to be construed as limiting the present invention.

### Example 1

### NMR-Derived Model of IP01

Materials and Methods

Reagents and Monoclonal Antibodies

Cysteine-constrained peptides, synthesized by Biopeptide (San Diego, Calif., USA), were isolated using high-performance liquid chromatography, and their correct mass was confirmed by mass spectrometry. The peptides were dissolved in 5% dimethylsulfoxide in phosphate-buffered saline (PBS) at concentrations of 10 mm and stored at −80° C. until use. The mAb directed against LFA-1 (TS1/18) was purified from a hybridoma line (American Tissue Culture Collection, Manassas, Va.) as described previously (Oj, V. T. & Herzenberg, T. (1980) Immunoglobulin-producing hybrid celllines. In Selected Methods in Cellular Immunology (Mishell, B. B. & Shiigi, S. M.,eds), H. H. Freeman, New York; Larson, R. S., Brown, D. C. & Sklar, L. A.(1997) Retinoic acid induces aggregation of the acute promyelocytic leukemia cell lineNB-4 by utilization of LFA-1 and ICAM-2.Blood 90, 2747-2756; Rothlein, R. & Springer, T. A. (1986) The requirement for lymphocyte function associated antigen 1 in homotypic leukocyte adhesion stimulated by phorbol ester. J. Exp.Med. 163, 1132-1149).

Measurement of LFA-1: ICAM-1 Dependent Cell Aggregation

JY cells were maintained in RPMI-1640 medium supplemented with 10% fetal bovine serum at 37° C. in an atmosphere containing 5% CO₂ in air. The aggregation of JY cells, which is dependent on LFA-1 binding to ICAM-1, was determined as previously reported (Shannon, J. P., Silva, M. V., Brown, D. C. & Larson, R. S. (2001) Novel cyclic peptide inhibits intercellular adhesion molecule-1-mediated cell aggregation. J. Pept. Res. 58, 140-150; Larson, R. S., Brown, D. C. & Sklar, L. A.(1997) Retinoic acid induces aggregation of the acute promyelocytic leukemia cell lineNB-4 by utilization of LFA-1 and ICAM-2.Blood 90, 2747-2756; Rothlein, R. & Springer, T. A. (1986) The requirement for lymphocyte function associated antigen 1 in homotypic leukocyte adhesion stimulated by phorbol ester. J. Exp.Med. 163, 1132-1149). Briefly, JY cells were washed twice with serum-free media and resuspended at a concentration of 4×10⁵ cells/mL. Cells were then preincubated with various concentrations of peptides or mAb for 15 minutes at room temperature. A 50 μL aliquot of cells was seeded in each well of a 96-well flat-bottomed microtiterplate followed by the addition of either peptide or mAb to a final volume of 100 μL. Cells were allowed to aggregate at 37° C. in humidified air containing 5% CO₂. Within each well, the number of aggregates and the total number of free (single) cells were counted by inverted phase microscopy. Percent aggregation, P, was then determined as follows: P=100(1−F_{f}/Fᵢ), where F_{f} (Fᵢ) is the final (initial) number of free cells. Percent aggregation was then used to calculate percent inhibition, I, as follows: I=100(1−Pᵢ)/P_{c}, where Pᵢ (P_{c}) is the percent aggregation with modulatory peptide (control). The constants for 50% inhibition (IC₅₀) were derived from a linear fit to the percent inhibition at four concentrations (50 μm, 100 μm, 500 μm, and 1 mm) of inhibitory peptide. Each experiment was performed at least three times, with each condition performed in duplicate. The data are reported as the mean values and the SDs of each IC₅₀.

2D-NMR Acquisition and Processing

Nuclear magnetic resonance spectra were acquired on a Bruker Avance 500 MHz instrument using WATERGATE water suppression with a 2.5 mm probe equipped with 3-axis gradients. NMR spectra were obtained at 279.0 K from peptide samples at concentrations of 2.5-8.5 mm at pH 7.4 in PBS (prepared in H₂O) to which 10% D₂O was added to provide a lock nucleus. Chemical shifts are reported with respect to internal 1 mm Sodium 3-(Trimethylsilyl)-1-propane-d₆-sulfononate (DSS; Isotec, Miamisburg, Ohio, USA). The temperature was monitored and controlled to within ±0.1 K with a Bruker (Billerica, Mass., USA) BVT3000 temperature controller operating a liquid nitrogen reservoir. A NOESY pulse sequence, using 10 mixing times (50, 100, 150, 200, 300, 400, 500, 600, 700, and 800 ms) was used to analyze the time-dependence of the NOEs. A total correlation spectroscopy (TOCSY) sequence, with a mixing time of 65 ms, and a DQF-COSY sequence were performed for assignments. In all cases, 256-1024 free induction decay (FIDs) were collected containing 170-256 scans each over a 6009 Hz sweep width. These were zero-filled once in F1, apodized with a Gaussian weighting function (LB=−3 Hz, GB=0.05) in F2 and a cosine-squared function in F1, and Fourier-transformed with polynomial solvent elimination in both dimensions with the aid of nmrPipe software (Rothlein, R. & Springer, T. A. (1986) The requirement for lymphocyte function associated antigen 1 in homotypic leukocyte adhesion stimulated by phorbol ester. J. Exp.Med. 163, 1132-1149) and FELIX (Accelrys, Inc., San Diego, Calif., USA). ³J_{HN-Hα} coupling constants were measured from high-digital resolution (0.1 Hz) one-dimensional spectra acquired with 64 K data points. Potential peptide backbone hydrogen-bonding was determined by examining the NH resonance positions in one-dimensional spectra taken every ˜5 K from 279 to 310 K. The temperature dependence of the amide proton chemical shifts was determined from linear least-squares fits to the data; the slopes are reported in p.p.b./K. These temperature-dependent experiments also showed that improved assignments of amide NOE connectivities could be made from two-dimensional spectra taken at 310 K. For this purpose, a ROESY spectrum at 310 K with a 175 ms spin-lock allowed us to assign the amide-amide Overhauser enhancement between R6 and M5, which is unresolved from S7 at 279 K.

2D-NMR Analysis and Restraint Generation

Nuclear Overhauser effect cross-peaks were assigned using FELIX. Where resolution allowed the assignment of symmetrical peaks in both dimensions, the weakest peak was used. NOE build-up curves of signal volume vs. mixing time were fitted to a quadratic polynomial. Approximate distance restraints were determined by comparing the volumes from cross-peaks of known distance (I8: |r(Hγ₁₁)−r(Hγ12)|=1.75 Å) to cross-peaks of unknown distance using the isolated spin-pair approximation (Farrar, T. C. & Becker, E. D. (1971) Pulse and Fourier Transform NMR: Introduction to Theory and Methods. Academic Press, New York.). Rather than using the simple classification of restraints into strong, medium, or weak, based on the NOE peak volumes, the higher precision of the distances derived from NOE build-ups enabled us to use the NOE-derived distances themselves as restraints. Upper and lower bounds of ±10% were introduced into these distances to accommodate spin-diffusion, conformational averaging and experimental uncertainties.

Geminal methylene protons and vicinal methyl-groups, while often resolved in the peptide spectra, were handled conservatively by replacing them in the structures with pseudoatoms, placed between relevant atom positions, rather than by presumptively assigning prochirality. Pseudoatom upper and lower restraint bounds were adjusted within the defaults of the NMR Architect module of Insight II, (Accelrys, San Diego, Calif., USA) as previously described (Wuthrich, K., Billeter, M. & Braun, W. (1983) Pseudo-structures for the 20 common amino acids for use in studies of protein conformations by measurements of intramolecular proton-proton distance constraints with nuclear magnetic resonance. J. Mol. Biol. 169, 949-961).

³J_{HN-Hα} coupling constants were measured in high resolution one-dimensional NMR experiments. Where spectral overlap occurred at 279 K, a series of higher temperature spectra (284-310 K) were examined and the coupling constants of resolved amide peaks were measured and extrapolated back to 279 K using linear least-squares fits to the data. For L2, L3, 18, and C9, the temperature-dependent slopes of the least squares lines were not significantly different from zero; however, the coupling constant for the amide proton from R6 increased from 6.4 Hz at 279 K to 8.2 Hz at 310 K. Peptide bond dihedral angles were determined from the coupling constants, and their error ranges, using the Karplus relationship: ³J=(6.8 Hz)cos²θ−(1.2 Hz)cos θ+2.5 Hz, as determined empirically for the H-N-Cα-Hα dihedral angle in proteins (Pardi, A., Billeter, M. & Wuthrich, K. (1984) Calibration of the angular dependence of the amide proton-C alpha proton coupling constants, ³J_{HN} alpha, in a globular protein. Use of ³J_{HN} alpha for identification of helical secondary structure. J. Mol. Biol. 180, 741-751; Karplus, M. (1959) Contact electron-spin coupling of nuclear magnetic moments. J. Chem. Phy. 30, 11-15). Upper and lower bounds were created by adding ±5° to the calculated values to accommodate conformational averaging and measurement variance.

Molecular Modeling

Distance-geometry calculations were performed using the program DGII within the NMR Architect module of Insight II, with force constants for all restraints set to 100.00 kcal/(mol Å²). Bounds smoothing was conducted using a triangle inequality algorithm followed by a tetrangle inequality algorithm, with a convergence criterion of 0.001 Å, and 5000 iterations through all atom quadruples (Havel, T. F. (1991) An evaluation of computational strategies for use in the determination of protein structure from distance constraints obtained by nuclear magnetic resonance. Prog. Biophys. Mol. Biol. 56, 43-78). Prospective metrization (Havel, T. F. (1991) An evaluation of computational strategies for use in the determination of protein structure from distance constraints obtained by nuclear magnetic resonance. Prog. Biophys. Mol. Biol. 56, 43-78) in four dimensions was performed, followed by embedding (Crippen, G. M. & Havel, T. F. (1978) Stable calculation of coordinates from distance information. Acta Cryst. A34, 282-284; Crippen, G. M. & Havel, T. F. (1988) Distance Geometry and Molecular Conformation. Research Studies Press, UK) and majorization with a constant weighting scheme. Simulated annealing optimization of the embedded structures was carried out as described in the Insight II supporting documentation. A conjugate gradient minimization was then carried out with sufficient iterations to ensure root mean square (RMS) gradient convergence of 0.001 Å. This process was repeated to generate a total of 99 structures, which took ˜1 hour on a 400 MHz Silicon Graphics Octane-2 workstation.

Simulated annealing was performed on the 99 DG structures using the MD schedule program within Insight II, which generates output files to be run in the Discover engine. DG can only incorporate single, discrete dihedral angle restraints despite the fact that multiple discrete dihedral angle ranges are solutions of the Karplus relation for each measured coupling constant. Therefore, another simulated annealing run was performed on each structure in order to incorporate the possible multiple discrete ranges. In order to favor the angle ranges most congruent with the NOE derived distance restraints, the distance restraints were imposed earlier than the dihedral angle restraints.

The SA protocol was run for a total of 9 ps with a step size of 1 femtosecond (fs) using the CVFF forcefield. Charges were calculated with a distance dependent dielectric constant scaled by a factor of 1.0. Heating from 200 to 300 K occurred over the first 1000 fs, with distance and dihedral force constants maintained at 100 and 25 kcal/(mol Å²) respectively, in order to allow the NOE distance restraints to influence which of the multiple discrete dihedral φ-angle ranges were adopted. Over the next 3000 fs, the dihedral force constants were scaled up to 100 kcal/(mol Å²). During the next 2000 fs, the distance and dihedral force constants were maintained at 100 kcal/(mol Å²). Finally, the temperature was cooled to 0 K over 3000 fs. A final minimization via the conjugate gradient method using distance and dihedral angle restraints with force constants of 100 kcal/ (mol Å²) was performed to a convergence parameter of 0.001 Å RMSD. This procedure took ˜30 minutes on a 400 MHz Silicon Graphics Octane-2 workstation for all 99 structures.

Mole Fractions of the Families

The chemical shifts for each of the four conformational families generated by the molecular dynamics runs (A-D, see Results) were calculated and it was assumed that the observed chemical shifts were a linear combination of the shifts in each family: δⱼ=XᵢCᵢⱼ. Here δⱼ was the vector of observed shifts labeled by the proton at position j, Xᵢ was a vector consisting of the mole fractions of each family i, (i=A, B, C, D), and Cᵢⱼ was the matrix of calculated shifts, containing i rows and j columns. The shifts for each family were calculated from Protein Data Bank-format files corresponding to each of the four mean structures by means of Williamson's Total 2 algorithm (Asakura, T., Taoka, K., Demura, M. & Williamson, M. P. (1995) The relationship between amide proton chemical-shifts and secondary structure in proteins. J. Biomol. NMR 6, 227-236). The vector X was calculated by using Matlab (The MathWorks, Natick, Mass., USA) to perform a non-negative, least-squares fit to the data. The variance in the resulting calculated mole fractions was estimated by randomly truncating the number of shifts in the matrix C and recomputing the resulting vector X.

Results

2D-NMR

Sequence-specific Spin-system Identification

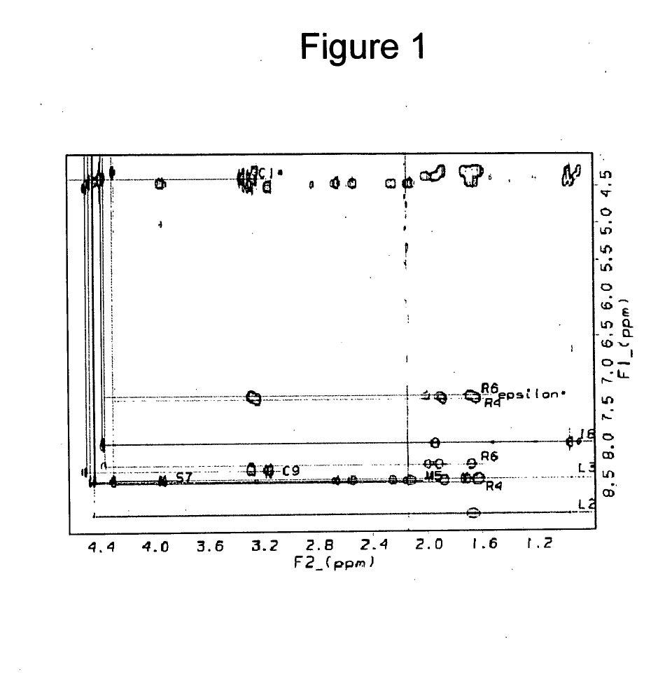

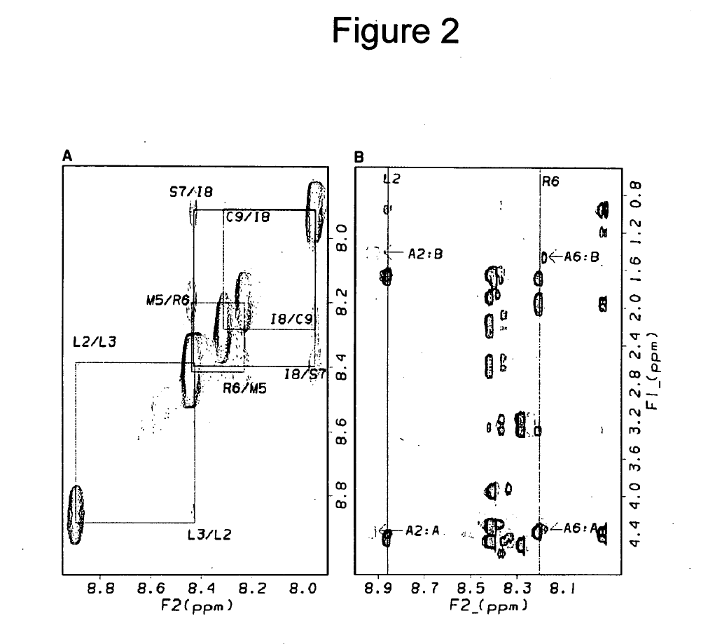

The spin-systems of each residue of IP01 (CLLRMRSIC; SEQ ID NO:2) were identified according to standard procedures (Wuthrich, K. (1986) NMR of Proteins and Nucleic Acids. J.Wiley and Sons, New York) in the ¹H TOCSY spectra (FIG. 1). The remaining task was to perform sequence-specific assignments to account for the three pairs of residues of IP01, which are duplicated, namely, C1 and C9, L2 and L3, and R4 and R6. These duplicated residues were assigned based on sequential amide proton NOEs and alanine substitutions. Sequential amide NOEs between 18 and one of the cysteines allowed assignment of the N- and C-terminal cysteines (FIG. 2A). This confirmed the features observed in the ¹H TOCSY and the one dimensional spectrum for the N-terminal cysteine (C1), which revealed a broad amide proton resonance near 8.5 p.p.m. without observable ¹H TOCSY cross-peaks, typical of a tertiary amine. Conversely, the C-terminal cysteine (C9), whose nitrogen was involved in a peptide bond, had a sharp doublet amide resonance in the one-dimensional spectrum and characteristic cross-peaks in the fingerprint region of the ¹H TOCSY spectrum. The remaining sequential amide NOEs confirmed the assignments for S7: 18, R :M5, and L: L, but were not informative in distinguishing L2 from L3 or R4 from R6 (FIG. 2A).

In order to perform sequence-specific assignments for the L2:L3 and R4:R6 spin-systems, ¹H TOCSY spectra were acquired from previously synthesized and reported alanine substituents of IP01 (FIG. 2B) (Vuorte, J., Lindsberg, P. J., Kaste, M., Meri, S., Jansson, S.-E., Rothlein, R. & Repo, H. (1999) Anti-ICAM-1 monoclonal antibody R6.5 (Enlimomab) promotes activation of neutrophils in whole blood. J. Immunol. 162, 2353-2357). The L2 to A2 substitution resulted in an amide chemical shift (8.92 p.p.m.) that was very similar to that found for one of the leucines in IP01 (8.86 p.p.m.), distinguishing the L2 amide proton from the L3 amide (8.40 p.p.m.). Sequence-specific assignment of R6 and R4 was performed by examining the NMR spectra from a peptide with an R6 to A6 substitution; the A6 amide chemical shift (8.21 p.p.m.) was very similar to that found for one of the arginines in IP01 (8.18 p.p.m.), but different from the other arginine amide resonance (8.42 p.p.m.), thus allowing both R4 and R6 to be to specifically assigned. The amide chemical shifts for a given residue in IP01 near the C1-C9 region appeared to depend on the residue's position in the sequence, rather than on the identity of the residue.

The chemical shifts for all of the protons in each sequentially assigned spin-system may be found in Table 1.

TABLE 1
Nuclear magnetic resonance (NMR) assignments for
inhibitory peptide-01 (IP01)

| | | | | δ (p.p.m.) | |
|---|---|---|---|---|---|
| | | | δ (p.p.m.) | random | Δδ |
| Position | Residue | Proton | experimental^{a} | coil^{b} | (p.p.m.) |
| 1 | CYS | HN | 8.5 | 8.53 | |
| | | | broad | | |
| | | Hα | 4.41 | 4.64 | −0.23 |
| | | Hβ | 3.35, 3.25 | | |
| 2 | LEU | HN | 8.86 | 8.27 | +0.59 |
| | | Hα | 4.45 | 4.36 + 0.09 | |
| | | Hβ | 1.69, 1.67 | | |
| | | Hγ | 1.64 | | |
| | | Hδ | 0.98, 0.94 | | |
| 3 | LEU | HN | 8.40 | 8.27 | +0.13 |
| | | Hα | 4.31 | 4.36 | −0.05 |
| | | Hβ | 1.65, 1.64 | | |
| | | Hγ | 1.61 | | |
| | | Hδ | 0.95, 0.88 | | |
| 4 | ARG | HN | 8.42 | 8.34 | +0.08 |
| | | Hα | 4.30 | 4.36 | −0.06 |
| | | Hβ | 1.88, | 1.87 | |
| | | Hγ | 1.64, 1.63 | | |
| | | Hδ | 3.25, 3.25 | | |
| | | Hε | 7.33 | | |
| | | Hξ | 7.01, 6.56 | | |
| 5 | MET | HN | 8.42 | 8.40 | +0.02 |
| | | Hα | .47 | 4.53 | −0.06 |
| | | Hβ | 2.26, 2.12 | | |
| | | Hγ | 2.66, 2.54 | | |
| | | Hδ | 2.15 | | |
| 6 | ARG | HN | 8.21 | 8.34 | −0.13 |
| | | Hα | 4.38 | 4.36 | +0.02 |
| | | Hβ | 2.01, 1.99 | | |
| | | Hγ | 1.70, 1.68 | | |
| | | Hδ | 3.29, 3.28 | | |
| | | Hε | 7.28 | | |
| | | Hξ | 7.01, 6.56 | | |
| 7 | SER | HN | 8.40 | 8.40 | +0.0 |
| | | Hα | 4.44 | 4.48 | −0.04 |
| | | Hβ | 3.96, 3.92 | | |
| 8 | ILE | HN | 7.93 | 8.14 | −0.21 |
| | | Ha | 4.39 | 4.19 | +0.20 |
| | | Hβ | 1.95 | | |
| | | Hγ | 11 1.52, 1.20 | | |
| | | Hγ | 2 | 0.96 | |
| | | Hδ | 0.90 | | |
| 9 | CYS | HN | 8.28 | 8.53 | −0.25 |
| | | Ha | 4.51 | 4.64 | −0.13 |
| | | Hβ | 3.29, 3.17 | | |

^{a}Chemical shifts are referenced to 3-(trimethylsilyl)-1-propane-d₆-sulfonic acid, sodium salt (DSS).
^{b}Random coil chemical shifts as reported by Plaxco et al. (Fairbrother, W. J., Christinger, H. W., Cochran, A. G., Fuh, G., Keenan, C. J., Quan, C., Shriver, S. K., Tom, J. Y., Wells, J. A. & Cunningham, B. C. (1998) Novel peptides selected to bind vascular endothelial growth factor target the receptor-binding site. Biochemistry 37, 17754-17764).

The unresolved one-dimensional resonances from the amide protons of L3, R4, M5, and S7 (˜8.40-8.42 p.p.m.) made it challenging to distinguish these signals even in two-dimensional spectra (FIG. 1). The remaining amide resonances (L2, R6, 18, and C9) were well resolved in both one- and two-dimensional spectra allowing for unambiguous assignments. The guanidinium protons of R4 and R6, at 7.33 and 7.28 p.p.m., respectively, were distinguished from their amide protons by their broader resonances, which coalesced at higher temperatures, indicative of rapid exchange with solvent protons, as well as by their ¹H TOCSY correlations with the broad guanidinium-group resonances at 7.01 and 6.56 p.p.m. The α-proton resonances from L2 to S7 were unresolved near 4.5 p.p.m. in one-dimensional spectra, but were distinguishable in the ¹H TOCSY spectrum. The resonances from the α-protons from L3 to R4 (˜4.30 p.p.m.) were indistinguishable at 279 K, but were resolved at 294 K. The remaining aliphatic resonances were initially distinguished by reference to standard nuclear shielding rules and by comparison with reference chemical shifts (Wuthrich, K. (1986) NMR of Proteins and Nucleic Acids. J.Wiley and Sons, New York). The few remaining ambiguities were clarified by examining ¹H DQF-COSY spectra, which aided the distinction of β- from γ-protons and γ- from δ-protons within each spin-system.

NOE Build-up and Distance Restraint Calculation

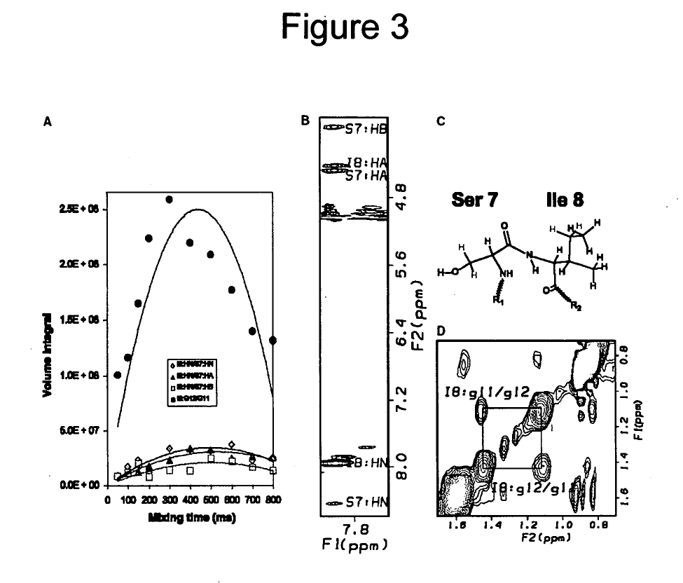

Seventy-eight resolved NOESY cross-peaks were identified and assigned for IP01. Fifty-two cross-peaks were part of a symmetric pair defined by the spectral diagonal. Of the symmetric peaks, only the weakest cross-peaks of each pair were used for restraint generation yielding a total of 52 useful peaks. Twenty of these peaks involved prochiral protons, which were handled conservatively through replacement by pseudoatoms, reducing this number by half and leaving a total of 42 distance restraints and one scalar peak for molecular modeling. Eight of these cross-peaks arose from inter-residue connections, while the remaining 35 cross-peaks arose from intraresidue connections. The volume of each cross-peak was determined for 10 mixing times (see Methods) to evaluate the rate of build-up (FIG. 3). The NOE cross-peak from the methylene protons of I8:Hγ₁₁-Hγ₁₂, which have a known covalent distance of 1.75 Å, was well resolved (FIG. 3D). The build-up of this cross-peak (FIG. 3A) was compared with those from the remaining cross-peaks to deduce the distances between their assigned protons. These distances and their associated upper and lower bounds (see Methods) are reported in Table 2.

TABLE 2
Nuclear Overhauser effect (NOE)-derived distance restraints

Restraint bounds^{a}

| | | | |
|---|---|---|---|
| Atoms | Lower | Upper | Distance^{b} |

Inter-residue

| | | | | |
|---|---|---|---|---|
| L2:HN | C1:Hb* | 2.14 | 2.54 | 2.38, 2.51 |
| L3:HN | L2:HN | 2.81 | 3.43 | 3.12 |
| M5:HN | R6:HN | 1.95 | 2.39 | 2.17 |
| I8:HN | S7:HN | 2.06 | 2.51 | 2.28 |
| I8:HN | S7:Ha | 2.24 | 2.74 | 2.49 |
| I8:HN | S7:Hb* | 3.13 | 4.83 | 3.48 |
| C9:HN | I8:HN | 2.33 | 2.85 | 2.59 |
| C9:HN | I8:Hb | 2.67 | 3.26 | 2.96 |

Intra-residue

| | | | | |
|---|---|---|---|---|
| L2:HN | L2:Ha | 2.00 | 2.44 | 2.22 |
| R4:HN | R4:Hb* | 2.18 | 3.66 | 2.42 |
| R4:HN | R4:Hc* | 2.41 | 3.95 | 2.68 |
| R4:H_{—} | R4:Ha | 3.49 | 4.27 | 3.88 |
| R4:H_{—} | R4:Hb* | 3.15 | 4.85 | 3.50 |
| R4:H_{—} | R4:Hc* | 3.10 | 4.79 | 3.44 |
| R4:H_{—} | R4:Hd* | 2.73 | 4.34 | 3.03 |
| R4:Ha | R4:Hb* | 1.94 | 3.37 | 2.16 |
| R4:Ha | R4:Hd* | 4.07 | 5.98 | 4.53 |
| R4:Hb* | R4:Hc* | 2.26 | 4.76 | 2.51 |
| M5:HN | M5:Hb* | 2.27 | 2.91 | 2.98, 2.52 |
| M5:HN | M5:Hc* | 2.42 | 2.85 | 2.69, 2.73 |
| M5:Ha | M5:Hb* | 2.22 | 2.70 | 2.46, 2.70 |
| M5:Ha | M5:Hc* | 2.29 | 2.66 | 2.55, 2.54 |
| R6:HN | R6:Hb* | 2.54 | 4.10 | 2.82 |
| R6:HN | R6:Hc* | 2.66 | 4.25 | 2.95 |
| R6:H_{—} | R6:Ha | 2.79 | 3.41 | 3.10 |
| R6:H_{—} | R6:Hb* | 2.82 | 4.45 | 3.13 |
| R6:H_{—} | R6:Hc* | 2.85 | 4.49 | 3.17 |
| R6:H_{—} | R6:Hd* | 2.75 | 4.36 | 3.05 |
| R6:Ha | R6:Hb* | 1.95 | 3.38 | 2.16 |
| R6:Ha | R6:Hd* | 2.54 | 4.11 | 2.83 |
| S7:HN | S7:Hb* | 2.62 | 4.20 | 2.91 |
| S7:Ha | S7:Hb* | 1.81 | 3.21 | 2.01 |
| I8:HN | I8:Ha | 2.45 | 2.99 | 2.72 |
| I8:HN | I8:Hb | 2.21 | 2.70 | 2.45 |
| I8:HN | I8:Hc1* | 2.39 | 2.96 | 2.96, 2.65 |
| I8:Ha | I8:Hc1* | 2.25 | 2.62 | 2.52, 2.50 |
| I8:Hb | I8:Hc1* | 2.32 | 3.08 | 2.58, 3.21 |
| I8:Hb | I8:Hc2* | 2.23 | 3.72 | 2.47 |
| I8:Hb | I8:Hd* | 2.70 | 4.30 | 3.00 |
| I8:Hc11 | I8:Hc12 | 1.57 | 1.93 | 1.75 |
| C9:HN | C9:Ha | 2.33 | 2.85 | 2.59 |
| C9:HN | C9:Hb* | 2.33 | 2.86 | 2.84, 2.59 |
| C9:Ha | C9:Hb* | 1.84 | 3.25 | −2.05 |

^{a}Upper and lower bounds were calculated as ±10% of the experimentally derived distance. Upper bounds of restraints to pseudoatoms were adjusted as per the Methods.
^{b}Distance is extrapolated from the NOE build-up curves using 10 mixing times (50-800 ms) using the NOE from I8:Hγ12-Hγ11 at 1.75 Å as described in the Methods.

³J_{HN-Hα} Coupling Constants and Dihedral Restraints

³J_{HN-Hα} coupling constants were measured for all resolved amide:Hα proton cross-peaks (L2, L3, R6, I8, and C9) in the ¹H one-dimensional spectra and were used to derive dihedral angle restraints to be used in molecular modeling. The coupling constants (ranging from 6.2 to 8.6 Hz; Table 3) were used to generate φ-dihedral angle restraints as described in the Methods. The multivalued Karplus relationship does not generally yield a unique solution for the angle φ (Table 3). Residue 18 had the highest ³J_{HN-Hα} coupling constant of 8.6 Hz, a value which yielded only two possible φ angle ranges, while residue C9's smaller ³J_{HN-Hα} coupling constant of 6.9 Hz was consistent with three φ-dihedral angle ranges. The remaining residues with measurable ³J_{HN-Hα} coupling constants (L2, L3, and R6) had the lowest values, ranging from 6.2 to 6.5 Hz, which resulted in four φ-dihedral angle ranges.

TABLE 3
³J_{HN-Hα} coupling constants

| Resi- | ³J_{HN-Hα} | Possible/-dihedral ranges^{b} | | | |
|---|---|---|---|---|---|
| due | (HZ)^{a} | 1 | 2 | 3 | 4 |
| C1 | — | — | — | — | — |
| L2 | 6.2 | 73 to 91 | −167 to −153 | −82 to −69 | 33 to 51 |
| L3 | 6.3 | 71 to 90 | −166 to −152 | −84 to −70 | 34 to 53 |
| R4 | — | — | — | — | — |
| M5 | — | — | — | — | — |
| R6 | 6.5 | 66 to 86 | −164 to −150 | −86 to −72 | 39 to 59 |
| S7 | — | — | — | — | — |
| I8 | 8.6 | −155 to −142 | −104 to −91 | — | — |
| C9 | 6.9 | −161 to −149 | −87 to −75 | 47 to 77 | — |

^{a}Experimental ³J_{HN-Hα} coupling constants are reported for nonoverlapped resonances in the amide region of the one-dimensional spectra as described in the Methods. The statistical errors in these are ±0.1 Hz.
^{b}Possible φ-dihedral ranges calculated from the ³J_{HN-Hα} coupling constants using the Karplus relationship as described in the Methods.

Structure Generation and Modeling

Distance-geometry calculations generated a series of 99 structures based on the NOE-derived distances and the ³J-derived dihedral angle restraints. These structures were then refined with simulated annealing and minimization as described in the Methods. Because of the high quality of the NOE-derived distance restraints, all structures with distance violations exceeding 0.1 Å were excluded, which left 82 structures for further analysis. All structures were confirmed to have the correct chirality and omega angle geometry after the simulated annealing runs. The RMSDs for the 82 structures were found to be 2.21 Å (backbone) and 4.38 Å (heavy atom); this large value of the heavy atom RMSD suggested that the molecule exists in multiple conformations.

Conformational Grouping

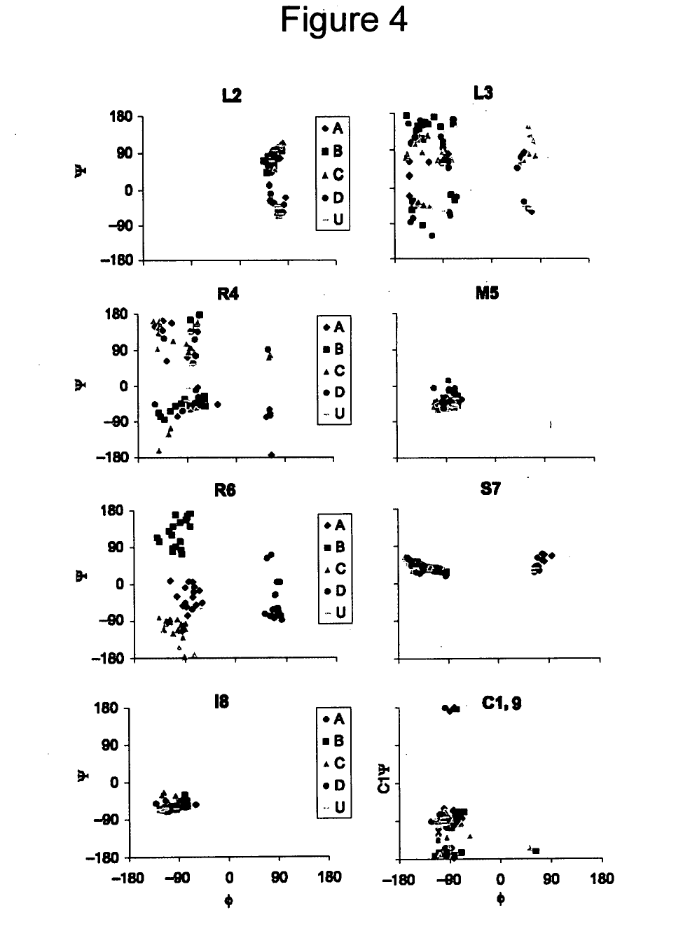

In order to determine the cause of the large RMSDs, the 82 structures were examined to ascertain whether multiple conformations indeed existed. Structures were classified using Ramachandran plots of the backbone dihedral angles φ and ψ (FIG. 4). The use of both NMR-derived restraints and molecular modeling yielded a single backbone geometry for M5, 18, and the φ angle of C9. Residues L2, S7 and the ψ angle of C1 were found in two possible backbone geometries. In the case of L2, a pathway of 180°ψ angle rotation about the cis-axis combined with a 45°φ angle variation was apparent. The pattern of the S7 plot was also suggestive, although less obvious, of a similar conversion process, but with a 180°φ angle rotation about the transaxis mediated by a 45°ψ angle variation. The ψ angle of C1 clustered at −90 and −180° for most structures. The conformations of residues L3, R4, and R6 favored defined negative φ values with a large distribution of ψ values. The backbone angles of S7 defined two distinct groups of structures, the majority (74%) of which had negative φ angles, while the remaining conformations (26%) had positive φ angles. The structures of S7 with φ<0 could be further classified into three distinct populations based on their clustering in the Ramachandran plot for R6, while the structures of S7 with φ>0 produced another distinctive region in the R6 plot. The backbone angular dependence of residues R6 and S7 did not correlate significantly with those of L3 or R4 so no further classification could be generated.

The result of this conformational analysis was that the 82 generated structures could be classified into four distinct families (A-D). Family A was composed of 17 structures which group in the S7φ>0, ψ>0 and R6φ<0, ψ<0 regions of the Ramachandran plot (FIG. 4). Four additional structures were within the S7φ>0, ψ>0 cluster, but had an apparently random distribution in the R6 plot, and thus were not classified (Class U in FIG. 4). Families B, C, and D were all related by their S7φ<0, ψ>0 backbone dihedral angles. Families B (n=19) and C (n=22) had R6 φ angles less than zero and were distinguished by their clustering with respect to the R6ψ˜120°: B, and R6ψ˜−135°: C regions. Close inspection of the data for R6 (FIG. 4) showed that the angular distribution of ψ for families B and C varied continuously over the range of ψ>90° to −90°<ψ, which was suggestive that B and C interconverted through a change in R6 ψ from 90° to −90° about the trans-axis. Family D, composed of 18 structures, was distinguished from families B and C by its R6 φ angle range φ>0°. Two additional structures were within the S7φ<0, ψ>0 cluster but did not fall within the above defined clusters in the R6 Ramachandran plot, and were thus left unclassified (U in FIG. 4).

This classification of the derived structures into families with differing conformations predicted that one might be able to observe these conformations using NMR, perhaps by varying the solvent composition. Several TOCSY and NOESY spectra of IP01 were examined at 279 K in mixtures of trifluoroethanol/water and methanol/water and indeed observed several alternate conformations. Nevertheless, conformation D was not observed in any of these solvent mixtures because a predicted NOE between the methyl protons on M5 with C1-Hα or C1-Hβ was not observed.

Energy Comparison of the Families

A comparison of the energies of each conformational family was made in order to determine if the relative enthalpies favored one conformational family over another. The energy of each structure in each family was calculated using three forcefields (CVFF, CFF91 and ESFF) (Asensio, J. L., Martin-Pastor, M. & Jimenez-Barbero, J. (1995) The use of CVFF and CFF91 force fields in conformational analysis of carbohydrate molecules. Comparison with AMBER molecular mechanics and dynamics calculations for methyl alpha-lactoside. Int. J. Biol. Macromol. 17, 137-148) in order to detect differences in their local energy minima. As none of the forcefields revealed any significant differences among the families, the results were based on those obtained using CVFF. The average energy, (E), and the SD for each family was: (E_{A})=−13.6±27.5; (E_{B})=0.5±37.4; (E_{C})=−8.8±25.8; and (E_{D})=10.5±51.1 kcal/mol. Because these values all existed within 25 kcal/mol of each other, and the smallest SD was 25.8 kcal/mol, these data did not energetically favor one conformation over another.

Backbone Chemical Shift and ³J_{HN-Hα} Analysis

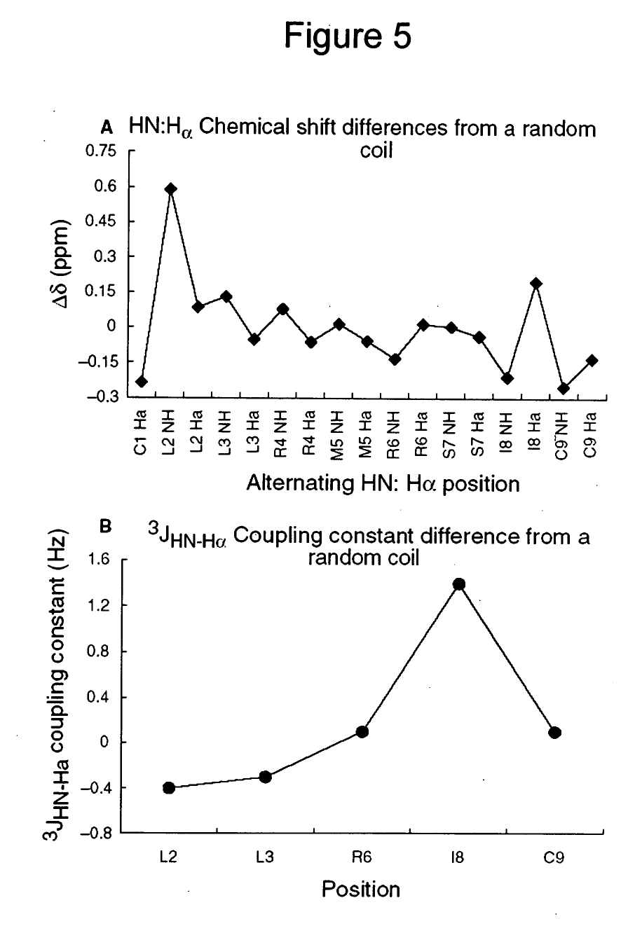

A comparison of the chemical shifts (Plaxco, K. W., Morton, C. J., Grimshaw, S. B., Jones, J. A., Pitkeathly, M., Campbell, I. D. & Dobson, C. M. (1997) The effects of guanidine hydrochloride on the ‘random coil’ conformations and NMR chemical shifts of the peptide series GGXGG. J. Biomol. NMR 10, 221-230) and the ³J_{HN-Hα} coupling constants (Smith, L. J., Bolin, K. A., Schwalbe, H., MacArthur, M. W., Thornton, J. M. & Dobson, C. M. (1996) Analysis of main chain torsion angles in proteins: prediction of NMR coupling constants for native and random coil conformations. J. Mol. Biol. 255, 494-506) of IP01 with values from random coils was useful for identifying regions with a defined backbone conformation (FIG. 5). The small chemical shift differences of up to ±0.15 p.p.m. observed for both the α- and amide-protons of residues L3, R4, M5, R6, and S7 were consistent with conformational averaging based on flexibility for this region of the molecule. Alternatively, the larger variations of >0.2 p.p.m. seen at positions C1, L2, I8, and C9 were indicative of a more defined, rigid region. The most prominent shift difference was seen for the amide proton of L2 (Δδ=0.59 p.p.m.). This large shift difference appeared to be a characteristic of the conformation of the peptide backbone at this position, as a similar shift difference (Δδ=0.65 p.p.m.) was observed when L2 was replaced by alanine (FIG. 2B). The α-proton of C1, the amide-proton of C9, and both the α- and amide-protons of 18 also showed large shift differences in the range of 0.20-0.25 p.p.m. In regions where spectral resolution was sufficient to measure amide coupling constants, the deviation at position I8 (Δ³J_{HN-Hα}=1.4 Hz) was the largest (FIG. 5B).

Comparison of the NMR chemical shift and coupling constant difference data with the backbone conformations derived from Ramachandran plots (FIG. 4) supported the molecular model. All conformations of the peptide backbone at residue L2 were unique because they all possessed a positive φ angle, a fact, which was congruent with the large observed chemical shift difference (Δδ_{HN}=+0.59 p.p.m.) at this position. For residue I8, the large difference in the coupling constant (Δ³J_{HN-Hα}=1.4 Hz) and the significant backbone chemical shift differences (Δδ_{HN}=−0.21 p.p.m. and Δδ_{Hα}=+0.20 p.p.m.) were consistent with a well-defined backbone configuration (FIGS. 4 and 5) at this site φ˜−110°, ψ˜−70°. Residue M5 also adopted a well-defined backbone configuration (FIG. 4; φ˜−90°, ψ˜−45°), however, an amide coupling constant for this position was unavailable because of spectral overlap. The large chemical shift differences for the cysteines (C1, Δδ_{Hα}=−0.23 p.p.m.; and C9, Δδ_{HN}=−0.25 p.p.m.) rounded out the evidence for the scaffold function of this structurally well-defined portion (I8-C9-C1-L2) of the peptide.

The temperature-dependent chemical shifts for the amide protons were determined over a series of seven temperatures ranging from 279 to 310 K using both one- and two-dimensional NMR experiments. Chemical shift changes in water less than ˜−3-4 p.p.b./K are indicative amide proton hydrogen bonding, whereas values of −10 p.p.b./K are typical of solvent exposed amide protons (Kessler, H. (1982) Peptide Conformations. 19. Conformation and biological-activity of cyclic-peptides. Angew Chem. Int. Ed. Engl. 21, 512-523). The temperature-dependence of our measured chemical shifts for IP01 ranged from −4.3 to −8.0 p.p.b./K, therefore hydrogen bonding among the amide protons could not be assumed. It is interesting to note, however, the large differences in slope observed for different residues; the NH protons from L2 and I8 had smaller slopes of 4.8 p.p.b./K, whereas the amide proton from L3 had a larger slope of −7.9 p.p.b./K. These results supported the notion that there were conformational differences among residues the peptide and that the structures were not random.

RMSD and Structure Description

As mentioned above, the 82 structures had a heavy atom RMSD of 4.4 Å and a backbone RMSD of 2.2 Å. The heavy atom RMSD was reduced through the process of grouping the structures into families to the range of 3.4-3.9 Å, while the backbone RMSD was similarly reduced to the range of 1.8-2.0 Å. Based on the defined families (FIG. 4), residues M5, R6, S7, and I8 had single, well-defined regions within their respective Ramachandran plots; therefore, RMSD calculations were generated for each family based only on these residues. The heavy atom RMSD decreased to 1.5-2.3 Å, while the backbone RMSD decreased to 0.7-0.9 Å for the four families. Residues C1, C9, and L2 were then added to the RMSD calculations. These positions were associated with more than one range of dihedral angle that was not accounted for by the family grouping, but were still tightly clustered, as opposed to the erratic spread seen for residues L3 and R4 (FIG. 4). The resultant RMSD calculations for residues C1, L2 and M5 to C9 yielded a range of 2.5-3.5 Å for the heavy atoms and 1.6-2.0 Å for the backbone atoms throughout the families.

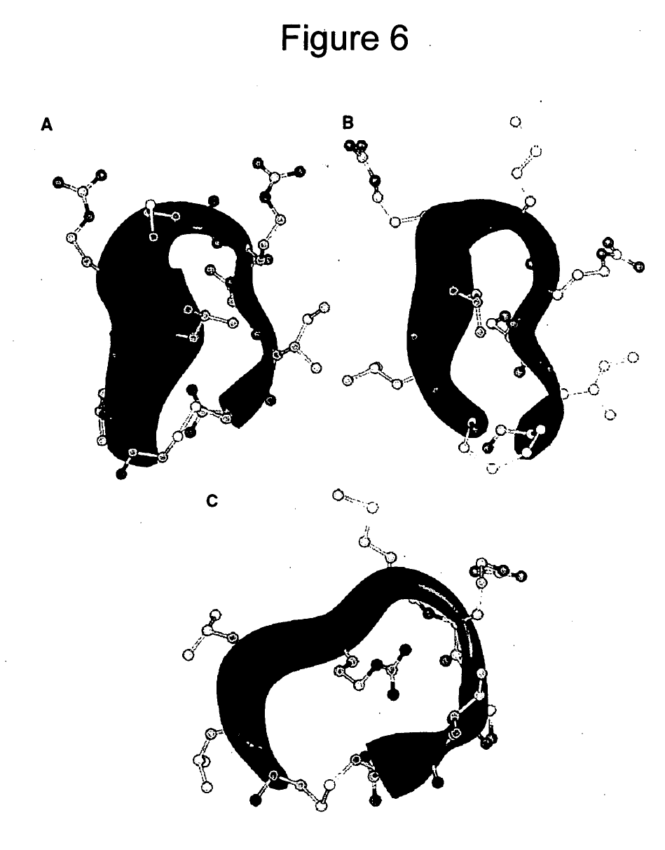

The lowest backbone RMSD structure of each family, relative to each family's average conformation, is shown in ball and stick format (FIG. 6). The backbone ribbon varies in width and color in order to encode into the figure the per-residue backbone RMSD. The M5 to I8 regions of each structure had a smaller RMSD than the C9 to R4 regions as a result of our structural classification based on the R6 and S7 dihedral angles, and because residues M5 and I8 had single well-defined clusters of backbone dihedral angles. Within the M5 to I8 region, the small RMSD (0.7-0.9 Å) of the backbone in all families supported a more detailed analysis.

To this end, a time-dependent study of each conformational family was performed to determine if entropic contributions, qualitatively noted by observing conformational motion in the analysis of the DG and SA results, existed to favor one conformational family over another. Each family was subjected to a low temperature (279 K) molecular dynamic simulation for 100 ps with the NMR-derived restraints imposed, using the lowest backbone RMSD structure of each family as the seed. Families A, B, and C were found to interconvert on a time scale of ˜10-15 ps. Family D did not interconvert with the other families during this simulation. Analysis of interconversion among families A, B, and C suggested the following pathway of interconversion: A⇄C⇄B where A and C, and C and B can interconvert, however, families A and B cannot interconvert directly, but must go through the common intermediate of C.

The relative mole fractions of each of the conformations A-D were determined. Our approach was to assume that the observed NMR spectrum was represented as a linear combination of the spectra from each of the families. The NMR spectrum from each of the families A-D was calculated (see Methods) and the resulting matrix equation (1) was solved to yield the following mole fractions of each family present in aqueous solution at 279 K: X_{A}=0.31±0.02; X_{B}=0.42±0.05; X_{C}=0.28±0.06; and X_{D}=0.01±0.01. These results further provided evidence that conformation D did not actually exist in solution.

Structural Features

Alanine Substitutions

Previously, alanine substitution and a cellular assay were used to define residues critical to the inhibitory activity of IP01 (Shannon, J. P., Silva, M. V., Brown, D. C. & Larson, R. S. (2001) Novel cyclic peptide inhibits intercellular adhesion molecule-1-mediated cell aggregation. J. Pept. Res. 58, 140-150). At that time it was reported that at least four residues (L2, L3, M5, and R6) were critical to the inhibitory function of IP01. While the R4 to A mutation resulted in an insoluble peptide that precluded further testing, the mutations of S7 and I8 to alanine yielded bioactivities within ±10% that of the lead inhibitor, a statistically insignificant difference in that assay. In order to quantify the effect of alanine substitutions, it was determined the IC₅₀ of each derivative in the cell aggregation assay as described in the Methods (Table 4). IP01 had an IC₅₀ of 530 μm at 2 hours. As was expected from previous aggregation assays, the IC₅₀ values of the L2, L3, M5, and R6 alanine substitutions were high (>1 mm). The S7 to alanine substitution produced an IC₅₀ of 740 μm. Interestingly, the I8 to alanine substitution had an IC₅₀ of 210 μm, which was a 2.5-fold improvement of inhibition over that found for IP01.

TABLE 4
Functional activity of inhibitory peptide-01 (IP01) alanine substitutions
(shaded residues)

| Peptide sequence^{a} | | | | | | | | | Peptide | |
|---|---|---|---|---|---|---|---|---|---|---|
| 1 | 2 | 3 | 4 | 5 | 6 | 7 | 8 | 9 | designation | IC₅₀ (IM)^{b} |
| C | L | L | R | M | R | S | I | C | IP01 | 530 |
| C | A | L | R | M | R | S | I | C | IP01-A2 | >1000 |
| C | L | A | R | M | R | S | I | C | IP01-A3 | >1000 |
| C | L | L | A | M | R | S | I | C | IP01-A4 | NS |
| C | L | L | R | A | R | S | I | C | IP01-A5 | >1000 |
| C | L | L | R | M | A | S | I | C | IP01-A6 | >1000 |
| C | L | L | R | M | R | A | I | C | IP01-A7 | 740 |
| C | L | L | R | M | R | S | A | C | IP01-A8 | 210 |

^{a}One letter amino acid code is used. N- and C-terminal cysteines are disulfide linked.
^{b}IC₅₀s were measured as described in the Methods section of the text. NS, not soluble.

Turn Analysis

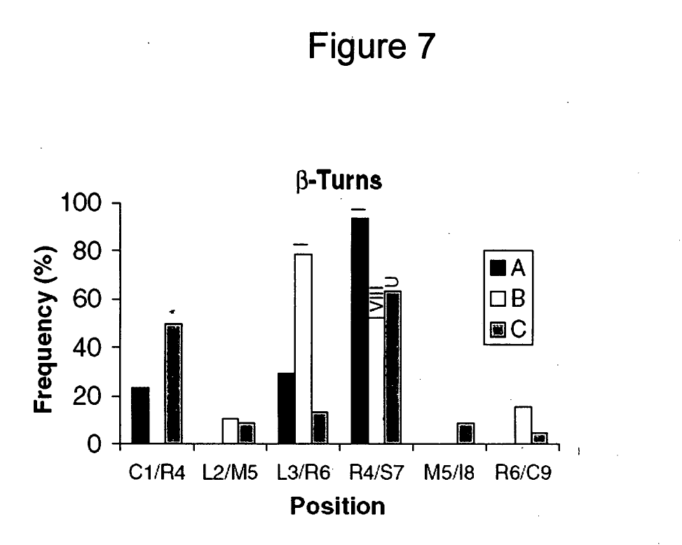

The conformational families A, B, and C were analyzed for the presence of β-turns because these motifs frequently occur at the binding sites of proteins. β-turns, defined by a maximum Cα₁-Cα₁₊₃ distance of 7 Å, were present in 90% of structures (FIG. 7). A β-turn involving residues R4-S7 was present in almost all (94%) of the structures in family A (FIG. 7). Family B had a dominant β-turn motif involving residues L3-R6 in 79% of structures and a less frequent β-turn involving residues R4-S7 in 56% of structures. The structure of family C was dominated by a β-turn involving residues R4-S7, found in 64% of structures, and a β-turn involving residues C1-R4 was seen in 50% of structures.

All significant turns were classified, when possible, by comparing the average dihedral angle of the group with published classifications. In family A, the R4-S7 β-turn was consistent with a type I turn (φᵢ₊₁=−74°, ψᵢ₊₁=−38°, φᵢ₊₂=−84°, ψᵢ₊₂=−30°) compared with published values of (−64°, −27°, −91°, −4°). Family B contained two classifiable β-turns, which were assigned as a type I turn (−86°, −46°, −76°, −42°) vs. (−64°, −27°, −91°, −4°) involving residues L3-R6, and a type VIII turn (−76°, −42°, −104°, 122°) vs. (−73°, −32°, 125°, 123°) involving residues R4-S7. Family C contained an unclassifiable β-turn involving residues R4-S7 (−81°, −43°, −106°, −109°).

Discussion

Structural and functional analysis has been performed on a cyclic peptide (IP01) inhibitor that binds to ICAM-1 and blocks binding to its counter-structure, LFA-1 (Shannon, J. P., Silva, M. V., Brown, D. C. & Larson, R. S. (2001) Novel cyclic peptide inhibits intercellular adhesion molecule-1-mediated cell aggregation. J. Pept. Res. 58, 140-150.). Several lines of evidence indicate that the cyclic peptide (IP01) adopts defined, non-random conformations. Foremost is the observation that opening of the disulfide bond destroys all bioactivity (Shannon, J. P., Silva, M. V., Brown, D. C. & Larson, R. S. (2001) Novel cyclic peptide inhibits intercellular adhesion molecule-1-mediated cell aggregation. J. Pept. Res. 58, 140-150). The NMR chemical shifts and coupling constants differ markedly from those found for a random coil. Well-defined β-turns exist in the calculated structures, which are also found in many other active peptide inhibitors. Finally, the restrained molecular simulations show that the backbone dihedral angles form well-defined clusters in Ramachandran plots.

The most critical IP01 residues for the inhibition of ICAM-1 binding were contained within the L2-L3-R4-M5-R6 region of the molecule (Table 4). Substitution of alanine for each of the residues L2,L3,M5, and R6 abrogated bioactivity, with IC₅₀s >1 m . Replacement of R4 by alanine resulted in an insoluble product. The NMR results indicated that these residues were conformationally flexible in solution. Alanine substitution for residues I8 and S7 produced a peptide which retained bioactivity, indicating that the C1-C9-I8-S7 region of the molecule was less critical from a functional standpoint.

Differential flexibility in the region of the critical functional residues, L2-R6, of IP01 was supported by the finding that the modeled backbone dihedral angles tended to vary over a larger range of values here than for the remainder of the molecule (FIG. 4), with the exception of methionine. In addition, the dihedral angles of residue L2 were found to lie in an unusual φ>0 region of the Ramachandran plot (FIG. 4) which indicated strain at this position; the most favored φ values are normally negative for amino acids. As this was an unusual conformation, the observed difference from a random coil peptide was significantly nonzero for both the chemical shift (Δδ=0.59 p.p.m.) and the coupling constant (Δ₃JHN-Hα=0.4 Hz). That this strained conformation was independent of the nature of the amino acid side chain at this site was indicated by the fact that replacement of L2 with alanine also resulted in a large chemical shift difference (Δδ=0.65 p.p.m.). Large, nonrandom, chemical shift differences have also been observed at this position for other cyclic, cysteine-constrained peptides. For example, a cyclic dodecapeptide (CVNENGGCEQYC (SEQ ID NO:9) from coagulation factor VII also showed a large shift difference (Δδ=0.65 p.p.m.) for the amide proton from V2 (Hu, C. K., Agner, K. E., Orning, L., Sakariassen, K. S., Stephens, R. W., Llinas, M. & Fischer, P. M. (2001) Synthesis, biological activity, and solution structures of a cyclic dodecapeptide from the EGF-2 domain of blood coagulation factor VII. J. Pept. Res. 57, 462-472.). Residues L3 and R4 also showed a great deal of flexibility; their backbone dihedral angles were predominantly found in the energetically favorable region where φ<0, and there were insignificant differences of the chemical shifts and coupling constants from random coil values at these positions. Residue M5, unlike the other residues on this side of the molecule, had a well-defined conformation in the favorable, negative/angle region of the Ramachandran plot. Because there is no structurally unique feature at this site, however, the chemical shift difference from a random coil was insignificant (a coupling constant was not measurable at this site). However, replacement of M5 with alanine abolished bioactivity (Table 4). The variability of the backbone dihedral angles for residue R6 (FIG. 4) indicated that a high degree of flexibility existed at this site. Most of the R6 conformations clustered into the negative/angle region (FIG. 4) with the exception of the conformations composing the unobserved family D. The chemical shift and coupling constant difference from the random coil was minimal for residue R6, in keeping with its high degree of flexibility.

Residues C1-C9-I8-S7 must play a less-critical role in support of IP01's inhibitory activity because alanine substitutions for residues I8 or S7 produce peptides which retained significant bioactivity (Table 4). Residue S7 was not critical to binding, and its backbone dihedral angles fell within two defined areas φ<0 and φ>0 of the Ramachandran plot, with the majority of structures falling in the preferred φ<0 range. This was consistent with the observation of small differences in the chemical shifts from random coil values (FIG. 5) and suggested that the structure at S7 is relatively rigid. Substitution of alanine for isoleucine at position 8 resulted in a twofold improvement of activity (Table 4) suggesting that the replacement of the bulky I8 side chain with the alanine methyl removed steric constraints at this position which allowed the backbone to assume a more favorable structure. The dihedral angles for I8 occupied a single defined region of the Ramachandran plot. The significant difference, from the random coil value, in the chemical shift (Δδ=0.21 p.p.m.) and the coupling constant (Δ3_{JHN-Hα}=1.4 Hz) supported our interpretation of relatively greater rigidity at this position. Residues C1 and C9 were unlikely to have significant functional relevance because their side chains were covalently linked and unavailable for interaction with the ligand. Their predominantly structural role was supported by the finding of a single φ dihedral angle region for residue C9 (FIG. 4) and a large chemical shift difference of Δδ=0.25 p.p.m. (FIG. 5); residue C1 had two defined w angle regions (FIG. 4) and a significant chemical shift difference of Δδ=0.23 p.p.m. (FIG. 5).

Turn identification has become a key component in peptide structural analysis, largely because several synthetic strategies for nonpeptide-based organic replacement of these moieties have been described and are useful for making second-generation inhibitors. Tight turns frequently mediate protein-protein interactions (Karawajczyk, B., Wirkus-Romanowska, I., Wysocki, J., Roka, K., Mackiewicz, Z., Glosnicka, R. & Kupryszewski, G. (2001) Cyclic analogue of human heat shock protein fragment. Synthesis, conformational studies and evaluation of its immunogenicity. Polish. J. Chem. 75, 265-273, Chen, C., Hsu, C. H., Su, N. Y., Lin, Y. C.,Chiou, S. H. & Wu, S. H. (2001) Solution structure of a Kunitz-type chymotrypsin inhibitor isolated from the elapid snake Bungarus fasciatus. J. Biol. Chem. 276, 45079-45087). The β-turn, defined by a Cai-Cai+3 distance of <7 Å (Chou, K. C. (2000) Prediction of tight turns and their types in proteins. Anal. Biochem. 286, 1-16.), is common among small cyclic peptides. As a mediator of protein-protein interaction, particularly in adhesion, it is likely that a β-turn within IP01 mediates the inhibitory function. Therefore, the observation of several β-turns among the different IP01 conformational families is in agreement with other findings with respect to inhibitory peptides (Leprince, J., Oulyadi, H., Vaudry, D., Masmoudi, O., Gandolfo, P., Patte, C., Costentin, J., Fauchere, J. L., Davoust, D., Vaudry, H. & Tonon, M. C. (2001) Synthesis, conformational analysis and biological activity of cyclic analogs of the octadecaneuropeptide ODN. Design of a potent endozepine antagonist. Eur. J. Biochem. 268, 6045-6057. Tamamura, H., Sugioka, M., Odagaki, Y., Omagari, A., Kan, Y., Oishi, S., Nakashima, H., Yamamoto, N., Peiper, S. C., Hamanaka, N., Otaka, A. & Fujii, N. (2001) Conformational study of a highly specific CXCR4 inhibitor, T140, disclosing the close proximity of its intrinsic pharmacophores associated with strong anti-HIV activity. Bioorg. Med. Chem. Lett. 11, 359-362. Brauer, A. B., Kelly, G., McBride, J. D., Cooke, R. M., Matthews, S. J. & Leatherbarrow, R. J. (2001) The Bowman-Birk inhibitor reactive site loop sequence represents an independent structural beta-hairpin motif. J. Mol. Biol. 306, 799-807., Bogusky, M. J., Culberson, J. C., Pitzenberger, S. M., Garsky, V. M., Wallace, A., Pessi, A. & Koblan, K. S. (1999) Conformation of a novel tetrapeptide inhibitor NH2-D-Trp-D-Met-Phe(pC1)-Gla-NH2 bound to famesyl-protein transferase. J. Pept. Res. 54, 66-73., Xu, C. R., Yusuf-Makagiansar, H., Hu, Y., Jois, S. D. & Siahaan, T. J. (2002) Structural and ICAM-1-docking properties of a cyclic peptide from the I-domain of LFA-1:an inhibitor of ICAM-1/LFA-1-mediated T-cell adhesion. J. Biomol. Struct. Dyn. 19, 789-799., Kanyalkar, M., Srivastava, S. & Coutinho, E. (2001) Conformation of N-terminal HIV-1 Tat (fragment 1-9) peptide by NMR and MD simulations. J. Pept. Sci. 7, 579-587). All three families (A, B, and C) have a high frequency of a β-turn involving residues R4-S7; this portion of the molecule contained three of the six functionally relevant residues.

### Example 2

### Solution Structure of IP02-K6

Materials and Methods

Reagents and Monoclonal Antibodies

Cysteine-constrained peptides, synthesized by Biopeptide (San Diego, Calif., USA), were dissolved at concentrations of 10 mM in 5% dimethylsulfoxide in phosphate-buffered saline, pH 7.4, and stored at −80° C. until use. The peptides were isolated using high-performance liquid chromatography, and the correct mass was confirmed by mass spectrometry. Some of the batches of peptide, while having identical mass spectra and chromatograms, nevertheless showed minor NMR conformers (10-30%) in the M5-C9 region of the molecule. Since the activities of the peptides in the cell aggregation assay could be entirely accounted for by the major fraction in each batch, these minor conformers were considered inactive and were not treated further. The mAb directed against LFA-1 (TS 1/22) was purified from a hybridoma line (American Tissue Culture Collection, Manassas, Va., USA) as described (Oj, V. T. and Herzenberg, T. (1980) in Selected Methods in Cellular Immunology. (Mishell, B. B. and Shiigi, S. M., Eds.) W.H. Freeman, New York).

Measurement of LFA-1:ICAM-1 Dependent Cell Aggregation

The same general procedure was used as described in Example 1.

NMR Data Acquisition and Processing

Acquisition and processing of 2D NMR data is described above. Briefly, the peptide samples were run at 279.0 K at concentrations of 2.5-8.5 mM in phosphate-buffered saline at pH 7.4 in H₂O to which 10% D₂O was added to provide a lock nucleus. Chemical shifts are reported with respect to internal 1 mM DSS (Sodium 3-(Trimethylsilyl)-1-propane-d₆-sulfonic Acid; Isotec, Miamisburg Ohio). The NMR spectra were acquired on a Bruker Avance 500 MHz instrument using WATERGATE water suppression with a 2.5 mm probe equipped with 3-axis gradients. The temperature was monitored and controlled to within ±0.1 K with a Bruker BVT3000 temperature controller operating a liquid nitrogen reservoir. The NOESY time courses were measured with mixing times ranging from 50 to 800 ms. Both DQ-COSY and total spin correlation spectra (TOCSY, mixing time=65 ms ) were measured and Fourier-transformed with polynomial solvent elimination in both dimensions with the aid of nmrPipe software (Delaglio, F., Grzesiek, S., Vuister, G. W., Zhu, G., Pfeifer, J., and Bax, A. (1995). NMRPipe: a multidimensional spectral processing system based on UNIX pipes. J. Biomol. NMR 6, 277-293) and Felix (Accelrys, Inc., San Diego, Calif.).

NMR Spectral Analysis and Restraint Generation

The mixing-time dependences of the NOEs were determined from quadratic polynomial fits to NOE peak volumes. Interproton distances were generated by comparing the volumes of cross-peaks from the γ₁₁ to the γ₁₂ protons in isoleucine (r=1.75 Å) to cross-peaks of unknown distance using the isolated spin-pair approximation (Farrar, T. C. and Becker, E. D. (1971) Pulse and fourier transform NMR: Introduction to theory and methods. Academic Press, New York). Geminal methylene protons and vicinal methyl-groups, while often resolved in the NOESY spectra, were handled conservatively in the modeling, by replacing them with pseudoatoms. Coupling constants (³J_{HN-Hα}) were measured in high digital resolution (0.1 Hz) 1D-NMR experiments. The temperature-dependence of the amide-proton chemical shifts was determined from linear least-squares fits of the shifts derived from several ID and TOCSY experiments at seven temperatures between 279 and 310 K. The line slopes are reported in ppb/K, with standard deviations ranging from 0.06-0.28 ppb/K.

Molecular Modeling

Distance geometry calculations were performed using Insight II (Accelrys, San Diego Calif.). The input for the calculations was [1] the molecular structure of IP01 minimized to a convergence of 10⁻⁷ using the conjugate gradient method, [2] the NOE-derived distance restraints, and [3] the ³J_{HN-Hα} coupling constant-derived dihedral angle restraints. Optimization of the structures was carried out with a short (10 ps) simulated annealing protocol. This process was repeated to generate 99 structures for further analysis, which took ˜1 hour on a 400 MHz Silicon Graphics Octane2 workstation: The solvent accessible surface was calculated, using water with a molecular radius of 1.4 Å, by the method of Lee and Richards (Lee, B. and Richards, F. M. (1971). The interpretation of protein structures: estimation of static accessibility. J. Mol. Biol. 55, 379-400) and Shrake and Rupley (Shrake, A. and Rupley, J. A. (1973). Environment and exposure to solvent of protein atoms. Lysozyme and insulin. J. Mol. Biol. 79, 351-371) used in Insight II.

IP02-K6:ICAM-1 Docking

Receptor Preparation: The structure of ICAM-1 from 1IC1.pdb (Casasnovas, J. M., Stehle, T., Liu, J. H., Wang, J. H., & Springer, T. A. (1998). A dimeric crystal structure for the N-terminal two domains of intercellular adhesion molecule-1. Proc. Natl. Acad. Sci. USA 95, 4134-4139) was assigned hydrogens and relaxed in the CVFF force field using Discover (Accelrys, Inc., San Diego, Calif.). In addition to the ordered waters present in the structure, the protein was soaked in five layers of explicit solvent. Added water and hydrogens were oriented with Steepest Descent and Conjugate Gradient minimizations to a derivative of 0.25 kcal·mol⁻¹·Å⁻¹ followed by dynamics for 6 ps in 1 fs steps. This was followed by Steepest Descent and Conjugate Gradient minimizations of the entire complex using a backbone tethering restraint that was slowly reduced until all of the atoms were free to move. The final derivative criterion was 0.10 kcal·mol⁻¹·Å⁻¹. The stereochemical quality of the proteins was examined using ProCheck software (Laskowski, R. A., MacArthur, M. W., Moss, D. S., & Thornton. J. M. (1993). PROCHECK: a program to check the stereochemical quality of protein structures. Journal of Applied Crystallography 26, 283-291). RMSD fitting was performed using the McLachlan algorithm (McLachlan, A. D. (1982). Rapid Comparison of Protein Structures. Acta Crystallographica Section A-Foundations of Crystallography 38, 871-873) as implemented in ProFit.

3D-Dock: The LFA-1 binding domain of ICAM-1 (residues 1-84 from pdb file IMQ8 (Shimaoka, M., Xiao, T., Liu, J. H., Yang, Y., Dong, Y., Jun, C. D., McCormack, A., Zhang, R., Joachimiak, A., Takagi, J., Wang, J. H., & Springer, T. A. (2003). Structures of the alpha L I domain and its complex with ICAM-1 reveal a shape-shifting pathway for integrin regulation. Cell 112, 99-111)) and the IP02-K6 ligand were prepared according to the specified protocol (removal of all hydrogens, water, and assignment of internal parameters). Default parameters were used to generate complexes in FT-Dock (Gabb, H. A., Jackson, R. M., & Sternberg, M. J. (1997). Modelling protein docking using shape complementarity, electrostatics and biochemical information. J. Mol. Biol. 272, 106-120). All 10,000 conformations generated by FT-Dock were subjected to local refinement with MultiDock (Jackson, R. M., Gabb, H. A., & Sternberg, M. J. (1998). Rapid refinement of protein interfaces incorporating solvation: application to the docking problem. J. Mol. Biol. 276, 265-285).

AutoDock: Partial Charges were assigned to the receptor and the ligand based on the CVFF forcefield. Non-polar hydrogen atoms were removed and charges were merged into the bonded carbon atoms. Electrostatic and potential grids were centered on the LFA-1:ICAM-1 binding site as identified in IMQ8.pdb (Shimaoka, M., Xiao, T., Liu, J. H., Yang, Y., Dong, Y., Jun, C. D., McCormack, A., Zhang, R., Joachimiak, A., Takagi, J., Wang, J. H., & Springer, T. A. (2003). Structures of the alpha L I domain and its complex with ICAM-1 reveal a shape-shifting pathway for integrin regulation. Cell 112, 99-111) and were set to encompass a volume of 2.7 μm³. For the rigid docking, all free torsions were removed and the docking was carried out using AutoDock 3.0 software (Morris, G. M., Goodsell, D. S., Halliday, R. S., Huey, R., Hart, W. E., Belew, R. K., & Olson, A. J. (1998) Automated Docking Using a Lamarckian Genetic Algorithm and Empirical Binding Free Energy Function. Journal of Computational Chemistry 19, 1639-62.53) with a population size of 50, 5×10⁵ energy evaluations, and 200 runs. For the semi-rigid docking, torsions were allowed in all rotatable bonds outside of the IP02-K6 backbone. The semi-rigid docking was carried out using a population size of 50, 1.5×10⁶ energy evaluations, and 800 runs. All calculations were performed on a SGI Origin running Irix 6.5 and using eight 300 MHz processors. CPU times are reported for a single processor only.

Results

Identification of Potent IP01 Derivatives.

Previously, Shannon et al. (Shannon, J. P., Silva, M. V., Brown, D. C., & Larson, R. S. (2001). Novel cyclic peptide inhibits intercellular adhesion molecule-1-mediated cell aggregation. J. Pept. Res. 58, 140-150) used semi-quantitative bioassays of IP01, and additional peptides containing alanine in place of single residues, and indicated that four of the nine residues in IP01 (L2, L3, M5, R6) were critical to binding, because the alanine-substituted peptides were poorer inhibitors. However, replacement of I8 with alanine resulted in a peptide (IP02) displayed enhanced inhibition. In order to improve structural and functional knowledge of IP02, single homologous amino acid substitutions of IP02 were undertaken with the goal of optimizing inhibitory function. The hydrophobic residues (L2, L3, I8) were substituted with the homologous amino acids I, L, and V. The hydrophilic residues (R4, R6) were substituted with K. M5 was also critical for binding (vide supra) but M had no amino acid homologue.

The functional activity of each IP02 derivative was measured in a slightly-modified version of the previously described cell aggregation assay (Shannon, J. P., Silva, M. V., Brown, D. C., & Larson, R. S. (2001). Novel cyclic peptide inhibits intercellular adhesion molecule-1-mediated cell aggregation. J. Pept. Res. 58, 140-150; Merchant, S. H., Gurule, D. M., Larson, R. S. (2003). Amelioration of ischemia-reperfusion injury with cyclic peptide blockade of ICAM-1. Am. J. Physiol. Heart Circ. 284,H1260-1268; Larson, R. S., Brown, D. C., and Sklar, L. A. (1997). Retinoic acid induces aggregation of the acute promyelocytic leukemia cell line NB-4 by utilization of LFA-1 and ICAM-2. Blood 90, 2747-2756; Rothlein, R. and Springer, T. A. (1986). The requirement for lymphocyte function-associated antigen 1 in homotypic leukocyte adhesion stimulated by phorbol ester. J. Exp. Med. 163, 1132-1149). Longer incubations (360 minutes) were used here instead of the earlier 120 minute incubations in order to increase the observed differences among peptide antagonists. Of all the amino acid substitutions, replacement of R6 with K, to produce IP02-K6, improved the IC₅₀ the most. Its IC₅₀ of 90 μM was ˜6-fold smaller than that found for the lead peptide, IP01 (IC₅₀=580 μM; Table 5). Similarly, changing L2 to I (IP02-12) or R4 to K (IP02-K4) resulted in improved inhibition with IC₅₀s between 105 and 190 μM. Alternatively, replacement of L2 or L3 with V (IP02-V2 and IP02-V3) or A8 with L (IP02-L8) abrogated activity, with IC₅₀s exceeding 1,000 μM. Finally, replacement of L3 with I (IP02-13) or A8 with V (IP02-V8) resulted in diminished but not complete abrogation of activity (IC₅₀s ˜700 μm).

TABLE 5

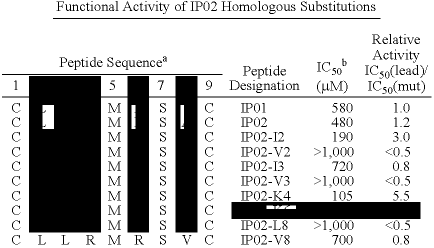

^{a}One-letter amino acid code. The N- and C-terminal cysteines are disulfide linked. Shading represents key positions identified via alanine substitution.
^{b}IC₅₀s were calculated as described in the Methods.

2D-NMR and Structure Generation

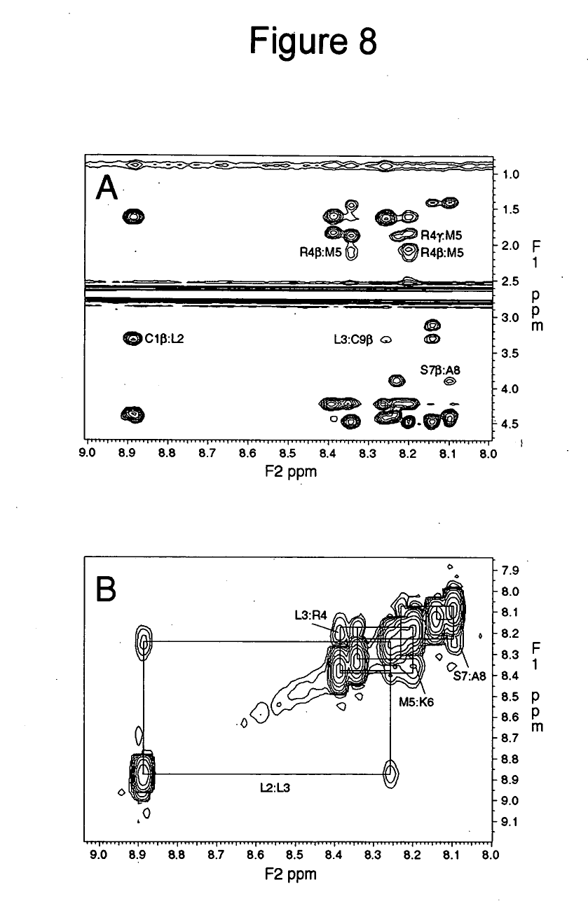

With the exception of the two residues which differ between the two peptides, the patterns and chemical shifts of the proton NMR spectra from IP02-K6 were generally similar to those previously described for IP01 so that the assignment of the IP02-K6 NMR spectrum was straightforward (FIG. 8). The IP02-K6 sequence-specific assignments for the duplicated residues C1/C9 and L2/L3 are identical to those for IP01, which were derived from sequential amide NOE connectivities and from comparisons with 2D-¹H TOCSY spectra of alanine subtitutions at the duplicated positions. The chemical shifts are reported in Table 6.

TABLE 6
Chemical Shift Assignments for IP02-K6

| | | | δ (ppm) | δ (ppm) | |
|---|---|---|---|---|---|
| Pos. | Residue | Proton | experimental^{a} | random coil^{b} | Δδ (ppm) |
| 1 | CYS | HN | broad | 8.53 | |
| | | Hα | 4.38 | 4.64 | −0.26 |
| | | Hβ1 | 3.28, 3.27 | | |
| 2 | LEU | HN | 8.89 | 8.27 | +0.62 |
| | | Hα | 4.44 | 4.36 | +0.08 |
| | | Hβ | 1.68, 1.66 | | |
| | | Hγ | 1.64 | | |
| | | Hδ | 0.97, 0.92 | | |
| 3 | LEU | HN | 8.23 | 8.27 | −0.04 |
| | | Hα | 4.26 | 4.36 | −0.10 |
| | | Hβ | 1.71, 1.67 | | |
| | | Hγ | 1.60 | | |
| | | Hδ1 | 0.94, 0.88 | | |
| 4 | ARG | HN | 8.39 | 8.34 | +0.05 |
| | | Hα | 4.24 | 4.36 | −0.12 |
| | | Hβ | 1.87, 1.85 | | |
| | | Hγ | 1.63, 1.60 | | |
| | | Hδ | 3.24, 3.21 | | |
| | | Hε | 7.29 | | |
| | | Hζ | 6.94, 6.54 | | |
| 5 | MET | HN | 8.16 | 8.40 | −0.24 |
| | | Hα | 4.51 | 4.53 | −0.02 |
| | | Hβ | 2.20, 2.08 | | |
| | | Hγ | 2.63, 2.53 | | |
| | | Hδ | 2.12 | | |
| 6 | LYS | HN | 8.36 | 8.35 | +0.01 |
| | | Hα | 4.22 | 4.32 | −0.11 |
| | | Hβ | 1.94, 1.89 | | |
| | | Hγ | 1.49, 1.45 | | |
| | | Hδ | 1.73, 1.70 | | |
| | | Hε | 3.03, 3.01 | | |
| | | Hζ | 7.61 | | |
| 7 | SER | HN | 8.24 | 8.40 | −0.16 |
| | | Hα | 4.39 | 4.48 | −0.09 |
| | | Hβ | 3.96, 3.90 | | |
| 8 | ALA | HN | 8.10 | 8.35 | −0.25 |
| | | Hα | 4.46 | 4.33 | +0.13 |
| | | Hβ | 1.45 | | |
| 9 | CYS | HN | 8.26 | 8.53 | −0.27 |
| | | Hα | 4.64 | 4.64 | 0.00 |
| | | Hβ | 3.37, 3.16 | | |

^{a}Chemical shifts are referenced to 3-(trimethylsilyl)-1-propane-d₆ sulfonic acid, sodium salt (DSS).
^{b}Random coil chemical shifts as reported by (Plaxco, K. W., Morton, C. J., Grimshaw, S. B., Jones, J. A., Pitkeathly, M., Campbell, I. D., and Dobson, C. M. (1997). The effects of guanidine hydrochloride on the ‘random coil’ conformations and NMR chemical shifts of the peptide series GGXGG. J. Biomol. NMR 10, 221-230)

NOE Build-Up and Distance Restraint Calculation.

One-hundred and six NOE cross-peaks were identified and assigned within the NOESY spectra. Thirty-six cross-peaks were members of a symmetric pair defined by the spectral diagonal. Of the symmetric peaks, only the weakest cross-peaks of each pair were used for restraint generation, leaving 88 remaining peaks. Sixty-six of these peaks involved prochiral protons, which were handled conservatively through the use of pseudoatoms (See Methods), thereby reducing this number by half and leaving a total of 55 distance restraints for NMR modeling. Four of these restraints were better approximated by covalent geometry, determined by distance geometry (DG) calculations, and were thus excluded from the modeling experiments, which was performed with the remaining 51 distance restraints. Twenty-two of these cross-peaks were inter-residual connections, while the remaining 29 cross-peaks were intra-residual connections. The distances derived from the NOE buildups and their associated upper and lower bounds (±10% of the NOE derived distance) can be found in Table 7.

TABLE 7
NOE-Derived Distance Restraints for IP02-K6

| | |
|---|---|
| Restraint Bounds^{a} | Measured |

| | | | |
|---|---|---|---|
| Atoms | Lower | Upper | Distance^{b} |

Inter-Residual

| | | | | |
|---|---|---|---|---|
| L2:HN | C1:HA | 2.2 | 2.7 | 2.5 |
| L2:HN | C1:HB* | 3.0 | 4.6 | 3.3 |
| L2:HN | C9:HB | 2.3 | 3.8 | 2.6 |
| L2:HN | L3:HN | 2.9 | 3.5 | 3.2 |
| R4:HN | L3:HD* | 5.4 | 7.6 | 6.0, 6.4 |
| R4:HN | M5:HN | 2.9 | 3.5 | 3.2 |
| M5:HN | R4:HA | 2.4 | 2.9 | 2.7 |
| M5:HN | R4:HB* | 2.9 | 3.5 | 3.4, 3.2 |
| M5:HN | R4:HG* | 3.0 | 3.7 | 3.6, 3.3 |
| M5:HN | K6:HN | 2.8 | 3.4 | 3.1 |
| K6:HN | M5:HA | 2.4 | 3.0 | 2.7 |
| K6:HN | M5:HB* | 3.4 | 4.4 | 4.5, 3.7 |
| S7:HN | A8:HN | 3.6 | 4.4 | 4.0 |
| S7:HA | R4:HB* | 5.1 | 6.6 | 6.6, 5,7 |
| S7:HA | R4:HG* | 3.7 | 4.6 | 4.1, 4.4 |
| S7:HB* | K6:HE* | 3.7 | 6.6 | 4.1 |
| A8:HN | S7:HA | 2.6 | 3.2 | 2.9 |
| A8:HN | S7:HB* | 3.2 | 3.9 | 3.7, 3.5 |
| A8:HA | S7:HB* | 4.0 | 5.1 | 4.9, 4.5 |
| C9:HN | A8:HA | 2.0 | 2.5 | 2.3 |
| C9:HN | A8:HB* | 3.4 | 5.2 | 3.8 |
| C9:HA | K6:HZ* | 4.9 | 7.0 | 5.5 |

Intra-Residual

| | | | | |
|---|---|---|---|---|
| C1:HA | C1:HB* | 2.0 | 2.6 | 2.3, 2.7 |
| L2:HN | L2:HA | 2.8 | 3.4 | 3.1 |
| L2:HN | L2:HD* | 4.4 | 7.8 | 4.9 |
| L2:HA | L2:HD* | 2.7 | 4.2 | 3.0, 3.3 |
| L3:HA | L3:HD* | 2.9 | 4.3 | 3.2, 3.3 |
| L3:HN | L3:HD* | 4.1 | 5.8 | 4.5 |
| R4:HN | R4:HB* | 2.5 | 3.0 | 2.7, 3.0 |
| R4:HA | R4:HB* | 2.7 | 3.2 | 3.0 |
| R4:HA | R4:HD* | 2.0 | 2.5 | 2.7, 2.2 |
| R4:HA | R4:HE | 3.1 | 3.8 | 3.5 |
| R4:HB* | R4:HE | 3.4 | 4.1 | 3.9, 3.7 |
| R4:HG* | R4:HE | 3.3 | 4.0 | 3.8, 3.7 |
| M5:HN | M5:HA | 2.6 | 3.1 | 2.8 |
| M5:HN | M5:HB* | 2.8 | 3.6 | 3.7, 3.1 |
| M5:HN | M5:HG* | 3.2 | 3.9 | 3.5, 3.7 |
| M5:HA | M5:HG* | 3.1 | 3.8 | 3.7, 3.4 |
| K6:HN | K6:HB* | 2.7 | 3.1 | 2.9, 3.0 |
| K6:HN | K6:HG* | 3.3 | 4.0 | 3.7, 3.7 |
| K6:HA | K6:HB* | 2.6 | 3.4 | 3.4, 2.9 |
| K6:HA | K6:HG* | 3.4 | 4.3 | 4.3, 3.7 |
| S7:HN | K6:HB* | 3.0 | 3.7 | 3.3, 3.6 |
| S7:HN | S7:HA | 2.6 | 3.2 | 2.9 |
| S7:HN | S7:HB* | 2.8 | 3.3 | 3.2, 3.1 |
| S7:HA | S7:HB* | 2.4 | 2.9 | 2.8, 2.7 |
| A8:HN | A8:HA | 2.7 | 3.2 | 3.0 |
| A8:HN | A8:HB* | 3.2 | 5.0 | 3.6 |
| C9:HN | C9:HA | 2.9 | 3.5 | 3.2 |
| C9:HN | C9:HB* | 2.7 | 3.4 | 3.3, 3.0 |
| C9:HA | C9:HB | 2.8 | 3.7 | 3.1, 3.8 |

^{a}Upper and lower bounds were calculated as ±10% of the experimentally derived distance. Upper bounds of restraints to pseudoatoms were adjusted as in Sillerud, et al. (2003).
^{b}Measured distance is computed from the NOE build-up curves using 10 mixing times (50 to 800 ms) as described in the Methods.

Backbone Chemical Shift and ³J_{HN-Hα} Analysis.

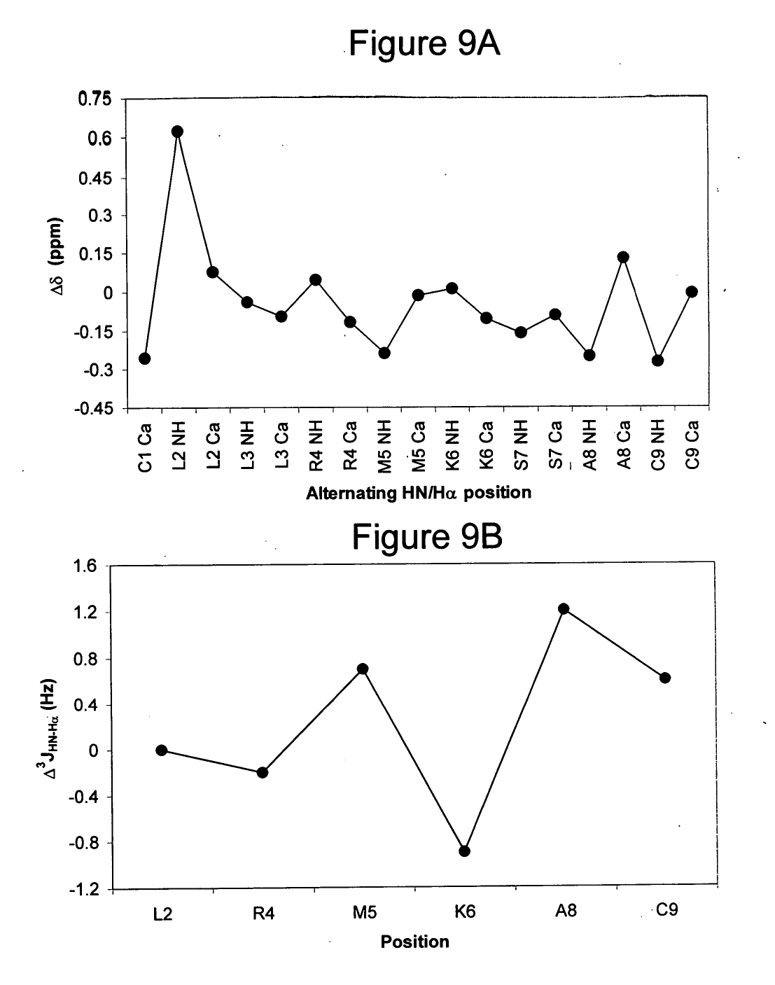

The NMR coupling constants, chemical shifts, and their temperature dependence, were used to identify regions of the peptide with nonrandom structures. A comparison of the differences between the random coil values for chemical shifts (Plaxco, K. W., Morton, C. J., Grimshaw, S. B., Jones, J. A., Pitkeathly, M., Campbell, I. D., and Dobson, C. M. (1997). The effects of guanidine hydrochloride on the ‘random coil’ conformations and NMR chemical shifts of the peptide series GGXGG. J. Biomol. NMR 10, 221-230) and ³J_{HN-Hα} coupling constants (Smith, L. J., Bolin, K. A., Schwalbe, H., MacArthur, M. W., Thornton, J. M., and Dobson, C. M. (1996). Analysis of main chain torsion angles in proteins: prediction of NMR coupling constants for native and random coil conformations. J. Mol. Biol. 255,494-506) and our measured values for IP02-K6 was used for identifying regions of this peptide possessing a well-defined backbone conformation (FIG. 9). Chemical shift differences of up to Δδ=±0.16 ppm were observed for both the α- and amide-protons of residues L3, R4, K6, and S7. Small shift differences like this are generally considered to reflect a random structure. However, variations of 0.2 ppm or greater were seen for the amide proton chemical shifts in positions C1, L2, M5, A8, and C9. The most prominent shift difference was observed for the amide proton of L2 with a Δδ of 0.62 ppm. Other significant shift differences, in the range of 0.24 to 0.27 ppm, were found for the α-proton of C1 and the amide protons of M5, A8, and C9.

The difference from a random coil for the coupling constants, Δ³J_{HN-Hα}, was most pronounced for the M5 through C9 segment of the molecule (ΔJ=0.6-1.2 Hz; FIG. 2B). Also noteworthy is the fact that the coupling constant difference was largest in the middle portion of this segment. Residues L2 and R4 showed only a small coupling constant difference of ΔJ<0.4 Hz, which was unlikely to be significant.

The temperature dependent chemical shift variation for each amide proton was determined over a series of seven temperatures ranging from 279 to 310 K using both 1D and 2D experiments. As a general rule, chemical shift changes in water that are <3-4 ppb/K are indicative of hydrogen bonding involving the amide protons, whereas values of 10 ppb/K are typical of a solvent exposed amide proton (Kessler, H. (1982). Peptide conformations.19. Conformation and biological-activity of cyclic peptides. Angew. Chem. Int. Ed. Engl. 21, 512-523). The temperature dependences of the chemical shifts for IP02-K6 were found to range from 5.2 to 6.2 ppb/K; therefore, strong hydrogen bonding among the amide protons was ruled out.

Structural Modeling and Features.

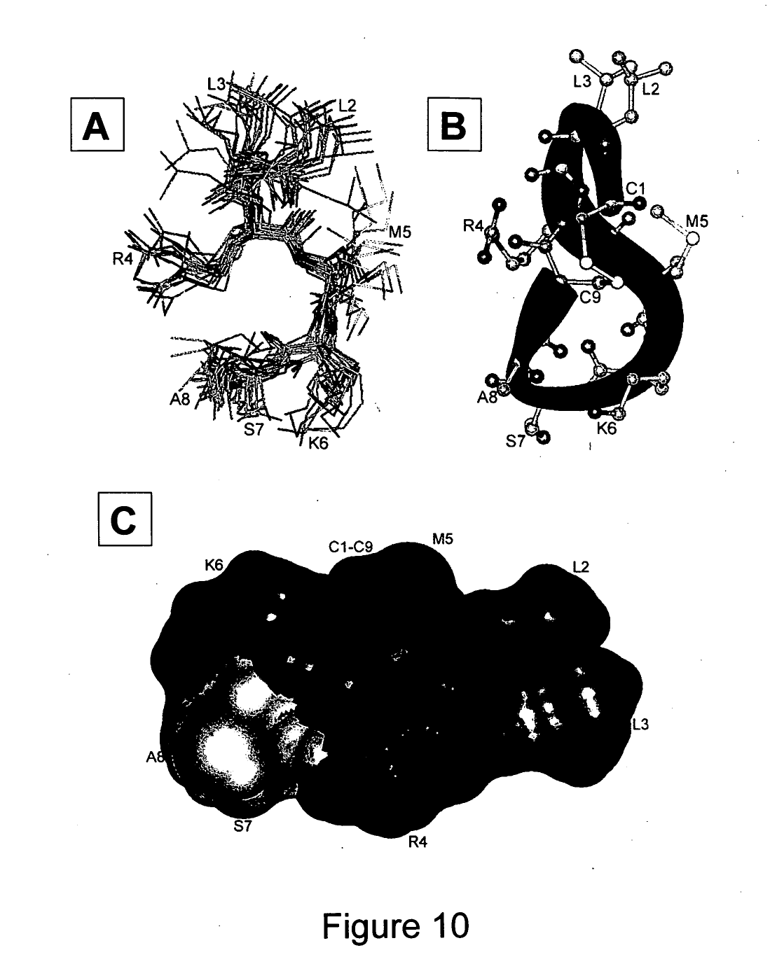

A series of 99 structures was generated using distance-geometry calculations based on the NOE-derived distances and ³J-derived dihedral angle restraints. These calculations were refined with simulated annealing and energy minimization. All structures were examined for restraint violations as well as for chirality and omega angle correctness; all structures were confirmed to have the correct chirality and omega angle geometry after the simulated annealing runs. Distance restraint violations did not exceed 0.5 Å and dihedral angle violations did not exceed 5° for any structure. By taking advantage of the high quality of the NOE-derived distance restraints, one structure with distance violations exceeding 0.2 Å was excluded leaving 98 structures for further analysis. A comparison of the energies of these structures with their RMSDs relative to the lowest energy structure revealed a single group of 16 low energy, low RMSD structures distinct from the remaining structures (FIG. 10A). RMSD calculations for these 16 structures revealed excellent convergence with a side chain atomic RMSD of 2.0 Å and a backbone atomic RMSD of 1.2 Å. This analysis showed that IP02-K6 adopted unique structural characteristics in solution. The backbone exists in a skewed S-shaped conformation with the lower lobe of the S-shape being the largest and encompassing residues R4-M5-K6-S7-A8-C9 (FIG. 10B). The upper lobe of this S-shaped structure is small and involves residues C1-L2-L3. This S-shaped structure is important in the docking of IP02-K6 onto ICAM-1 (vide infra).

Turn Analysis.

Because β-turn motifs frequently occur at the binding sites of proteins (Chou, K. C. (2000). Prediction of tight turns and their types in proteins. Anal. Biochem. 286, 1-16) all 16 of the conformations of IP02-K6 were analyzed for the presence of this turn. A β-turn is defined by a maximum Cα₁-Cα₁₊₃ distance of 7 Å. Turns were found to be common in the lower lobe of the S-shaped structure of IP02-K6, but were rare in the upper lobe. In particular, a β-turn involving residues R4-M5-K6-S7 is noted in half of the structures. This β-turn occurred in a region of limited conformational flexibility with average dihedral angle values of φᵢ₊₁=−133°, ψᵢ₊₁=−96°, φᵢ₊₂=−88°, ψᵢ₊₂=−50°, values which have not been previously described (Leprince, J., Oulyadi, H., Vaudry, D., Masmoudi, O., Gandolfo, P., Patte, C., Costentin, J., Fauchere, J. L., Davoust, D., Vaudry, H., and Tonon, M. C. (2001). Synthesis, conformational analysis and biological activity of cyclic analogs of the octadecaneuropeptide ODN. Design of a potent endozepine antagonist. Eur. J Biochem. 268, 6045-6057) as a unique β-turn type, but which are closest to a type-I β-turn (φᵢ₊₁=−60°, ψᵢ₊₁=−30°, φᵢ₊₂=−90°, ψᵢ₊₂=0°).

Solvent Accessible Surface.

The model of IP02-K6 produced by the combination of NMR results and distance-geometry has a low RMSD for both the backbone (1.2 Å) and the side chains (2.0 Å) allowing for a detailed analysis of the solvent accessible surface (FIG. 10C). The total solvent exposed area for IP02-K6 was 1118 Å², 74% (822 Å²) of which was formed by the residues critical to function (L2, L3, R4, M5, K6, A8). This critical region is predominantly hydrophobic, consisting of 73% non-polar (601 Å²) and 27% polar (221 Å²) areas.

Several observations were made about the surface presented by IP02-K6 to ICAM-1 for binding. The side chains of the hydrophobic residues L2 and L3 aligned closely together at one pole of the molecule, and the side chain from M5 folded towards this pole with its hydrophobic surface confluent with these residues and the disulfide linked C1 and C9 (FIG. 10). The side chains of the hydrophilic residues R4 and K6 folded to the opposite pole of the molecule; however, their surfaces never became confluent as in the case of L2, L3, and M5 because they were physically separated by the side chains of S7 and A8.

Docking of IP02-K6 with ICAM-1.

In order to ascertain if the solution conformation of IP02-K6 determined from the NMR data reflected the ICAM-1 bound conformation of IP02-K6, docking studies were performed using the structure of ICAM-1 determined by X-ray crystallography (1IC1.pdb) (Casasnovas, J. M., Stehle, T., Liu, J. H., Wang, J. H., & Springer, T. A. (1998). A dimeric crystal structure for the N-terminal two domains of intercellular adhesion molecule-1. Proc. Natl. Acad. Sci. USA 95, 4134-4139) and the solution structure of IP02-K6 presented here. Prior to docking, the ICAM-1 structure was relaxed to relieve instances of high energies due to inappropriate bond lengths, main-chain bond angles, or deviations from planarity (see Methods). The RMSD of the relaxed structure was 0.83 Å and only minor changes were seen in the ICAM-1 residues identified to be involved in LFA-1 binding (Jackson, R. M., Gabb, H. A., & Sternberg, M. J. (1998). Rapid refinement of protein interfaces incorporating solvation: application to the docking problem. J. Mol. Biol. 276, 265-285). Three strategies were considered for IP02-K6 docking due to the complexity of the problem. In the first, the 3D-Dock suite of programs, consisting of FT-Dock (Shimaoka, M., Xiao, T., Liu, J. H., Yang, Y., Dong, Y., Jun, C. D., McCormack, A., Zhang, R., Joachimiak, A., Takagi, J., Wang, J. H., & Springer, T. A. (2003). Structures of the alpha L I domain and its complex with ICAM-1 reveal a shape-shifting pathway for integrin regulation. Cell 112, 99-111), RPScore (Moont, G., Gabb, H. A., & Sternberg, M. J. (1999). Use of pair potentials across protein interfaces in screening predicted docked complexes. Proteins 35, 364-73), and MultiDock (Gabb, H. A., Jackson, R. M., & Sternberg, M. J. (1997). Modelling protein docking using shape complementarity, electrostatics and biochemical information. J. Mol. Biol. 272, 106-120) was used. This approach was designed for studying protein-protein interactions. Initial conformations for rigid docking were generated using FTDock for geometric surface recognition based on Fourier correlation theory. The docked conformations were then screened according to the residue pair-potential scoring function with RPScore. Finally, local refinement in a molecular mechanics force field was performed with full receptor and ligand flexibility (MultiDock). The other two approaches utilized AutoDock 3.0 (Morris, G. M., Goodsell, D. S., Halliday, R. S., Huey, R., Hart, W. E., Belew, R. K., & Olson, A. J. (1998) Automated Docking Using a Lamarckian Genetic Algorithm and Empirical Binding Free Energy Function. Journal of Computational Chemistry 19, 1639-62.53) software to perform either rigid-, or semi-rigid, docking in a molecular mechanics force-field using a genetic algorithm.

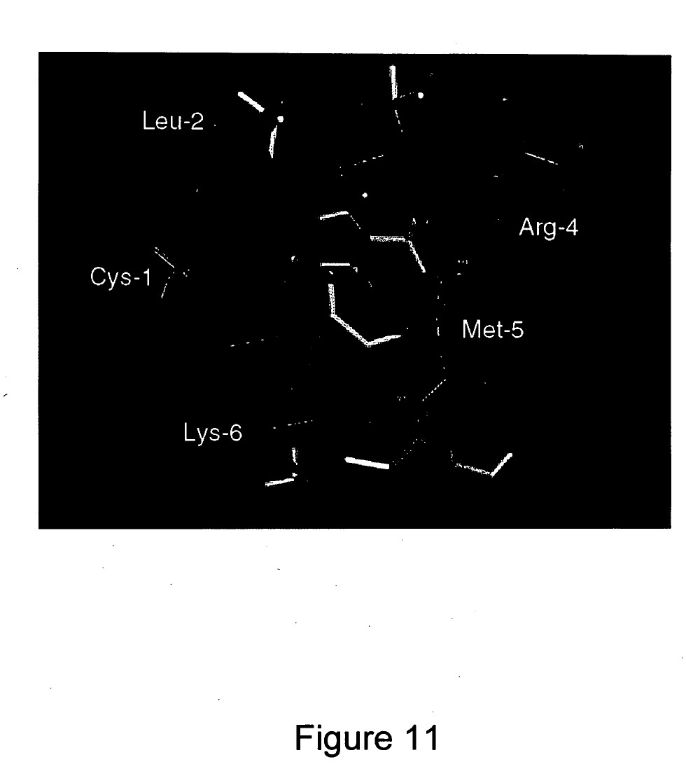

Of the three docking algorithms used, rigid-docking with AutoDock consumed the least computer time. It required about 3.5 CPU hours to attain 10% (20/200) of the total docked structures within 1 Å RMSD of the low energy conformation. Unfortunately, this also yielded a relatively high-energy complex structure (AutoDock Force Field: −4.3 kcal·mol⁻¹). Relaxation of the complex with local search via MultiDock improved the energy of the complex (MultiDock: −28.8 kcal·mol⁻¹). Docking using 3D-Dock required about 2.5 days (real time) to complete. This runtime included scoring of all 10,000 conformations generated by FT-Dock, because in this case, RPScore was found to lack efficacy in screening out complexes that would eventually yield high MultiDock energies. Despite the fact that the 3D-Dock approach was performed in a similar manner to that used by AutoDock (rigid docking followed by MultiDock refinement), this docked structure was considerably lower in energy (MultiDock: −50.5 kcal·mol⁻¹) due to the very large set of conformations that were subjected to local refinement. The minimum energy docked structure was generated by AutoDock semi-rigid docking, where side-chain flexibility was allowed outside of the IP02 backbone during the global minimization (FIG. 11). However, in order to have confidence that the global minimum was reached (11 out of the 800 total docked structures within 1 Å RMSD of the low energy conformation), 12 CPU days were required for run-time calculations.

The docked structure of IP02-K6 differed little from the solution state conformation (FIG. 11); the only significant changes resulted from movements in the R4 and K6 side chains (RMSD 1.79 Å). The solution structure of IP02-K6 was able to be docked onto ICAM-1 without changes in the peptide backbone from those observed in solution by NMR.

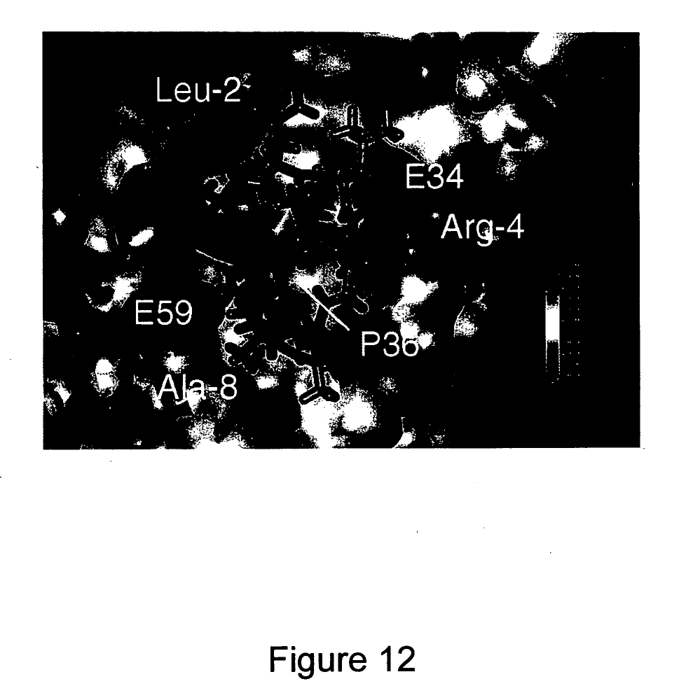

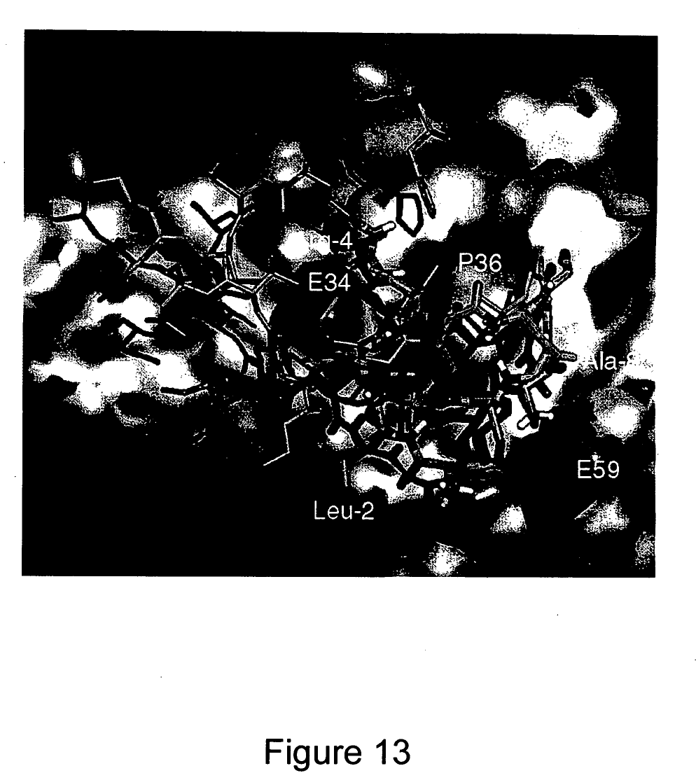

All of the docking approaches found the same binding site for their respective lowest-energy complexes, exhibiting interactions with ICAM-1 residues 33-39, 57-62, 64, 66 and 77. The minimum energy complex generated from AutoDock semi-rigid docking exhibited the most overlap with the LFA binding site (FIGS. 12 and 13). The IP02-K6:ICAM-1 binding interactions are summarized in Table 8.

TABLE 8
IP02-K6:ICAM-1 Binding Interactions

| | | | Contribution to |
|---|---|---|---|
| Position | Residue | ICAM-1 Binding Residues^{a,b} | Binding Energy^{c} |
| 1 | CYS | D60, S61, Q62, K77 | 6.2% |
| 2 | LEU | E34, K77 | 6.0% |
| 3 | LEU | E34, T35, P36, M64 | 9.7% |
| 4 | ARG | I33, E34, T35, P36, K39, Y66 | 22.6% |
| 5 | MET | P36 | 0.7% |
| 6 | LYS | P36, E59 | 10.0% |
| 7 | SER | P36, L37, P38 | 6.4% |
| 8 | ALA | P36, L37, V57, Q58, E59, S61 | 15.5% |
| 9 | CYS | T35, P36, L37, E59, S61, Q62 | 22.9% |

^{a}Based on a 4 Å cutoff from IP02-K6 atoms
^{b}Bold residues indicate interactions important to LFA-1 binding.
^{c}Based on van der Waals, hydrogen bond, and electrostatic contributions as calculated by the AutoDock 3.0 force field (Morris, G. M., Goodsell, D. S., Halliday, R. S., Huey, R., Hart, W. E., Belew, R. K., & Olson, A. J. (1998) Automated Docking Using a Lamarckian Genetic Algorithm and Empirical Binding Free Energy Function. Journal of Computational Chemistry 19, 1639-62.53).

Hydrogen bonding was observed between IP02-K6 residue R4 and ICAM-1 residues E34, T35, and K39, and between IP02-K6:C1 and K77. IP02-K6:K6 was held by electrostatic interactions with E59, while IP02-K6:A8 was held by tight van der Waals interactions with L37, V57, Q58 and E59 (FIGS. 12 and 13). These docking results provided insight into the relationship between bioactivity and homologous amino acid substitution at IP02-K6 position 8. The methyl group of alanine at position 8 was in van der Waals contact with the side chains of V57, Q58 and E59 from ICAM-1. Replacement of the methyl group of A8 of IP02-K6 with the bulkier side chains of either V, L or I significantly lowered the bioactivity of the resulting peptides (Table 5). There was a correlation between the volumes (Pontius, J., Richelle, J., & Wodak, S. J. (1996) Deviations from Standard Atomic Volumes as a Quality Measure for Protein Crystal Structures. J. Mol. Biol. 264,121-136) of the side chains of the residues at IP02-K6 position 8 and the resulting IC₅₀s (A, vol=92 Å³, IC₅₀=480 μM; V, 138 Å³, 700 μM; L, 163 Å³, >1000 μM) which likely resulted from sterically-forced repositioning of the important ε-NH₃⁺ on K6 away from the carboxyl group on E59 of ICAM-1 as the side chain volume at IP02-K6 position 8 was increased above that from alanine.

Discussion

The structural, functional, and NMR data from IP01, IP02-K6 and their derivatives served to identify the residues important for inhibitory function. The results obtained indicate that there are distinct, but overlapping, components that serve, on the one hand, as scaffolding, and on the other, as a functional inhibitory binding site in IP02-K6. Amino acid substitution studies indicated that the residues most important for inhibitory activity include the stretch from L2 to K6, while residues S7-C1 appear to be important in maintaining the structural and backbone integrity of the inhibitor. The NMR results and the turn analysis show a conserved β-turn between residues R4 and S7. Interestingly, prominent coupling constant and chemical shift differences from the random coil (FIG. 9) exist in the region of this β-turn, which supports the notion of a conserved structural motif at this location. The hydrophobicity and docking model support this division of the molecule into scaffolding and binding components.

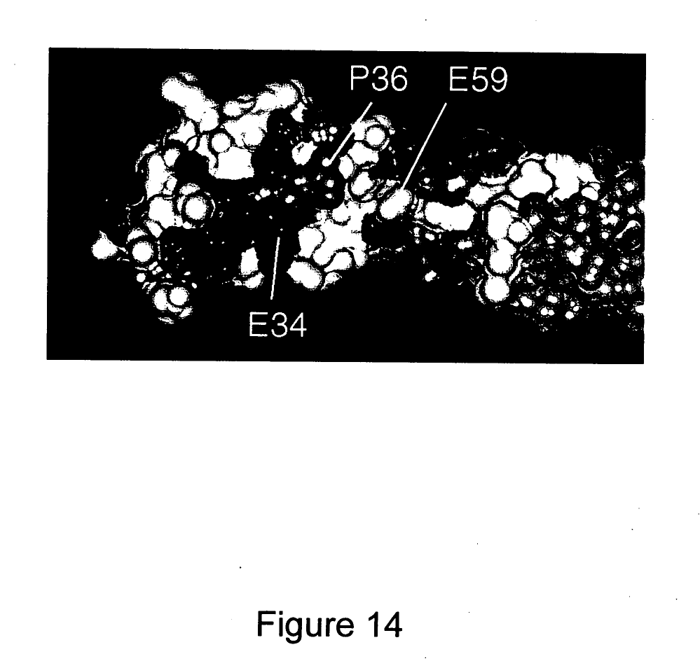

The site where LFA-1 binds to ICAM-1 was inferred by mapping the existing mutation data (Staunton, D. E., Dustin, M. L., Erickson, H. P., Springer, T. A. (1990). The arrangement of the immunoglobulin-like domains of ICAM-1 and the binding sites for LFA-1 and rhinovirus. Cell 61,243-254) onto the surfaces of the molecules (FIG. 14) and from the x-ray structure (1MQ8.pdb) of LFA-1 bound to ICAM-1 (Shimaoka, M., Xiao, T., Liu, J. H., Yang, Y., Dong, Y., Jun, C. D., McCormack, A., Zhang, R., Joachimiak, A., Takagi, J., Wang, J. H., & Springer, T. A. (2003). Structures of the alpha L I domain and its complex with ICAM-1 reveal a shape-shifting pathway for integrin regulation. Cell 112, 99-111). The analysis shown in FIG. 14 agrees with the work of Shimaoka, et al., (Jackson, R. M., Gabb, H. A., & Sternberg, M. J. (1998). Rapid refinement of protein interfaces incorporating salvation: application to the docking problem. J. Mol. Biol. 276, 265-285). IP02-K6 docks to this same region on ICAM-1 (FIG. 13). Comparison of the LFA-1 binding site on the surface of ICAM-1, with that of IP02-K6 reveals a region of high similarity (FIG. 12). The binding site is composed of residues from three strands of ICAM-1 (residues 33-39, 57-62, 64, 66, and 77). Hydrophilic interactions occur between ICAM-1 residue E34 and IP02-K6:K6 as well as between ICAM-1:K77 and the peptide backbone at IP02-K6:C1 (Table 6). Hydrophobic interactions are found between ICAM-1:M64 and IP02-K6:M5, and between L37, V57 and IP02-K6:A8. This latter interaction would be expected to be optimal given the small size of the alanine methyl group, but would be less favorable in the valine, leucine, and isoleucine derivatives of IP02-K6, which would explain the decrease in peptide bioactivity with the bulkier side-chain substitutions at this site (Table 5). IP02-K6 residues L2 and L3 are flexible and interact with ICAM-1 residues E34, K77, T35, P36 and M64 (Table 6). The solution structure of IP02-K6 (FIG. 10B) contains a β-turn in the region from R4-S7. This turn allows the backbone of IP02-K6 to drape over the side-chain from P36 in ICAM-1. In this model, all of the 7 ICAM-1 residues known to be critical to LFA-1 binding (FIG. 14) would be involved with peptide binding, explaining the mechanism of peptide inhibition.

Since IP02-K6 is less flexible than IP01, the improvement in IP02-K6 binding affinity, over that of IP01, may be attributed to a structure with reduced conformational entropy that reflects the ICAM-1 bound conformation. The differences between IP01 and IP02-K6 lie in the replacement of bulky, flexible side chains (R6, 18) with smaller side chains (K6, A8) leading to a more-compact, lower-entropy molecule.

Despite the large translation ranges that were allowed for ligand docking, all three docking approaches described herein predicted the same binding site. The minimum energy conformation of IP02 (based on comparison via MultiDock analysis) was obtained from semi-rigid docking in which side-chain flexibility was allowed. Despite the allowed torsions, the docked structure exhibited little difference from the solution structure (RMSD=1.79 Å). The only significant structural changes observed were movements in the R4 and K6 side chains allowing them to interact with E34 and E59. Additionally, within a 3.2 kcal·mol⁻¹ range from the minimum energy docked conformation consisting of the top 16 conformations generated from clustering, the RMSD from the minimum energy conformation did not exceed 2 Å, suggesting that only minor conformational differences occur within this energy range.

IP02-K6 shares many similarities with conformation C of IP01. A R4-S7 β-turn was present in 67% of structures in the IP01 family C and similarly in 45% of the structures in IP02-K6. Both of these turns have dihedral angle values that are similar (maximum dihedral angle variation of 60°) and have not been previously described in the classification of β-turns. These results provide a structural model of IP02-K6 inhibition of ICAM-1.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Various references are cited herein, the disclosures of which are referred to or incorporated by reference in their entireties.

1. A method of identifying a modulator of ICAM-1 activity by rational drug design comprising:
(a) providing a tertiary structure of CLLRMXaa₁SXaa₂C (SEQ ID NO:1), where Xaa₁ is R or K and Xaa₂ is I or A;
(b) employing the tertiary structure of (a) to select a potential modulator of ICAM-1, wherein said modulator binds to the ICAM-1 substrate binding site;
(c) obtaining said modulator and
(d) determining whether the potential inhibitor inhibits the activity of ICAM-1.

2. The method of claim 1, wherein the potential modulator is a non-peptide organic molecule.

3. The method of claim 1 wherein the potential modulator is designed to form hydrogen bonds with E34, T35, and K39 of ICAM-1.

4. The method of claim 1 wherein the potential modulator is designed to have hydrophilic interactions with E34 and K77 of ICAM-1.

5. The method of claim 1 wherein the potential modulator is designed to have hydrophobic interactions with M64, L37, and C57 of ICAM-1.

6. The method of claim 1, wherein the potential modulator comprises a β turn.

7. The method of claim 6, wherein the β turn has a maximum dihedral angle variation of 60°.

8. The method of claim 1, wherein the potential modulator comprises ICAM-1 binding properties of IP01 and IP02-K6.

9. The method of claim 1, wherein in step (a) the tertiary structure of CLLRMRSIC (SEQ ID NO. 2) is provided.

10. The method of claim 1, wherein in step (a) the tertiary structure of CLLRMKSAC (SEQ ID NO. 3) is provided.

11. The method of claim 1, wherein the tertiary structure in step (a) is generated using modeling techniques employing data in Tables 1, 2, 3, 6 or 7, or combination of the foregoing ±a root mean square deviation of not more than 2.0 Å from the backbone atoms of the amino acids of SEQ ID NO. 1.

12. The method of claim 11, wherein the tertiary structure in step (a) is generated using modeling techniques employing data in Tables 1, 2 and 3.

13. The method of claim 11, wherein the tertiary structure in step (a) is generated using modeling techniques employing data in Tables 6 and 7.

14. The method of claim 1, wherein step (b), employing the tertiary structure to designing the potential modulator comprises identifying a compound structurally similar to SEQ ID NO:1.

15. The method of claim 1, wherein in step (c) the potential modulator is obtained by synthesis.

16. A method of identifying modulators of ICAM-1 comprising:
(a) screening for substances having at least one of the characteristics selected from the group consisting of: forming hydrogen bonds with E34, T35, and K39 of ICAM-1, having hydrophilic interactions with E34 and K77 of ICAM-1, have hydrophobic interactions with M64, L37, and C57 of ICAM-1, comprises a β turn, and comprises ICAM-1 binding properties of IP01 and IP02-K6; and
(b) determining whether the substance screened in (a) modulates the activity of ICAM-1.

17. The method of claim 16, wherein the substance is a peptide.

18. The method of claim 16, wherein the substance is a non-peptide organic molecule.

19. A modulator of ICAM-1 activity identified according to the method of claim 1.

20. A modulator of ICAM-1 activity identified according to the method of claim 16.

21. A modulator of ICAM-1 activity having the following characteristics: forming hydrogen bonds with E34, T35, and K39 of ICAM-1, having hydrophilic interactions with E34 and K77 of ICAM-1, have hydrophobic interactions with M64, L37, and C57 of ICAM-1, comprises a β turn, and comprises ICAM-1 binding properties of IP01 and IP02-K6.

22. The modulator according to claim 21, wherein said modulator is a non-peptide organic molecule.

23. The modulator according to claim 21, wherein said modulator is a peptidomimetic.

24. A peptide variant of SEQ ID NO:1 having the sequence CXaa₁Xaa₂Xaa₃MXaa₄SXaa₅C, wherein Xaa₁ and Xaa₂ are L or I, wherein Xaa₃ and Xaa₄is R or K, Xaa₅ is A, L or I with the proviso that when Xaa₁ and Xaa₂ are L, Xaa₃ is R, Xaa₄ is R or K, Xaa₅ is L.

25. The peptide variant or SEQ ID NO:1 having the sequence selected from the group consisting of: CILRMRSAC (SEQ ID NO:4), CLIRMRSAC (SEQ ID NO:5), CLLKMRSAC (SEQ ID NO:6), CLLRMKSAC (SEQ ID NO:7), CLLRMRSLC (SEQ ID NO:8).

26. A composition comprising the modulator of claim 21 and a carrier.

27. The composition comprising the peptide variant of claim 24 and a carrier.

28. A method for treating an ICAM-1/LFA-1 mediated disease comprising administering an amount of the modulator of claim 21 to a subject in need thereof effective to treat said ICAM-1/LFA mediated disease.

29. A method for treating an ICAM-1/LFA-1 mediated disease comprising administering an amount of the composition of claim 26 to a subject in need thereof effective to treat said ICAM-1/LFA mediated disease.

30. A computer for producing a three dimensional representation of a molecule having the sequence CLLRMXaa₁SXaa₂C (SEQ ID NO:1), where Xaa₁ is R or K and Xaa₂ is I or A comprising
(a) computer-readable data storage medium comprising a data storage material encoded with computer-readable data, wherein said data in Tables 1, 2, 3, 6 or 7, or combination of the foregoing;
(b) a memory for storing instructions for processing said computer-readable data;
(c) a central-processing unit coupled to said working mememory and to said computer-readable data storage medium for processing said computer-machine readable data into said three-dimensional representation; and
(d) a display coupled to said central-processing unit for displaying said three-dimensional representation.
